(19) United States
(12) Patent Application Publication
(10) Pub. No.: US 2025/0368991 A1
(43) Pub. Date: Dec. 4, 2025

(54) OLIGONUCLEOTIDES

(71) Applicants: AGENCY FOR SCIENCE, TECHNOLOGY AND RESEARCH, Singapore (SG); THE JIKEI UNIVERSITY, Tokyo (JP)

(72) Inventors: Jin Rong OW, Singapore (SG); Eri IMAGAWA, Tokyo (JP); Tommaso TABAGLIO, Singapore (SG); Kimihiko OISHI, Tokyo (JP); Dave Keng Boon WEE, Singapore (SG)

(21) Appl. No.: 18/876,053

(22) PCT Filed: Jun. 28, 2023
(86) PCT No.: PCT/SG2023/050458

§ 371 Date: Dec. 17, 2024

(30) Foreign Application Priority Data
Jun. 28, 2022 (SG) 10202250320V

(51) Int. Cl. C12N 15/113

(52) U.S. Cl. CPC C12N 15/113; C12N 2310/11; C12N 2310/315; C12N 2310/321; C12N 2310/3231; C12N 2320/33

(57) ABSTRACT

The present invention relates generally to the field of RNA splicing. In particular, the invention relates to splice-switching oligonucleotides (SSOs) capable of altering the splicing of a pre-mRNA encoding a variant of the SLC25A13 gene. The invention also relates to the use of SSOs as therapeutic candidates for treating citrin deficiency. In an aspect of the invention, there is provided a method of exon-skipping comprising providing a splice-switching oligonucleotide (SSO) that binds to a site within a target region present on a pre-mRNA transcript of the SLC25A13 gene, wherein the binding of the SSO induces the exclusion of SLC25A13-PE5 from a mature mRNA transcript of the SLC25A13 gene. In another aspect, there is provided a splice-switching oligonucleotide (SSO) that binds to a site within a target region present on a pre-mRNA transcript of the SLC25A13 gene, the target region having at least 95% sequence identity to SEQ ID NO: 28, and wherein binding of the SSO induces the exclusion of SLC25A13-PE5 from a mature mRNA transcript of the SLC25A13 gene.

### FIELD OF THE INVENTION

The present invention relates generally to the field of RNA splicing. In particular, the invention relates to splice-switching oligonucleotides (SSOs) capable of altering the splicing of a pre-mRNA encoding a variant of the SLC25A13 gene. The invention also relates to the use of SSOs as therapeutic candidates for treating citrin deficiency.

### BACKGROUND

Citrin deficiency is an autosomal recessive disorder of urea cycle metabolism, caused by pathogenic variants in the SLC25A13 gene encoding citrin, a mitochondrial aspartate-glutamate carrier. This condition can manifest as neonatal intrahepatic cholestasis during infancy (NICCD) and citrullinemia type II (CTLN2) characterized by adult-onset recurrent hyperammonemia with altered mental status that is refractory to conventional hyperammonemia therapies. NICCD may usually be self-limited and it is followed by a relatively asymptomatic period during childhood. During the so-called “asymptomatic period”, some patients with citrin deficiency may have recurrent hypoglycemia, feeding difficulties, or growth restriction. Affected individuals generally have a unique eating pattern (preference of high-fat/protein foods and avoidance of high-carbohydrate diet) with typically lean habitus before the onset of CTLN2. Identifying at-risk individuals during this period is challenging due to the absence of specific clinical findings or biochemical markers to address the disease progression. Undiagnosed individuals are at risk for significant growth restriction, hypoglycemia, and sudden onset of life-threatening hyperammonemia. Therefore, establishing a diagnosis of citrin deficiency in a timely manner and the development of effective therapies are crucial. The detailed mechanism of the disease remains unclear.

Currently, the only available management for citrin deficiency is dietary modification with a high-protein/fat diet and adding medium chain triglycerides in diet in some cases. Additionally, high carbohydrate diet and alcohol intake are discouraged since these can cause metabolic decompensations including hyperammonemia, which can lead to neurological damages. However, while dietary modification may be used to manage the symptoms of citrin deficiency, it does not cure the underlying genetic cause of the disease. Recent studies suggested that the carrier frequency of citrin deficiency was relatively high (nearly 1 in 30-40), particularly in East Asian countries such as in Singapore or Japan. Thus far, no curative management for citrin deficiency exists except for liver transplantation. However, liver transplantation is a major operation that comes with risks of medical complications such as bleeding and infections. Liver transplantation also carries risks such as immune rejection, biliary complications, transplanted liver failure and the need for lifelong immune suppression medication. There is thus a need for a new therapeutic strategy that corrects the underlying pathogenic genetic variant of the SLC25A13 gene that overcomes the drawbacks of the prior art. Furthermore, other desirable features and characteristics will become apparent from the subsequent detailed description and the appended claims, taken in conjunction with the accompanying drawings and this background of the disclosure.

### SUMMARY

In one aspect, there is provided a method of exon-skipping comprising providing a splice-switching oligonucleotide (SSO) that binds to a site within a target region present on a pre-mRNA transcript of the SLC25A13 gene, wherein the binding of the SSO induces the exclusion of SLC25A13-PE5 from a mature mRNA transcript of the SLC25A13 gene.

In one embodiment, the target region has at least 95% sequence identity to SEQ ID NO: 28.

In one embodiment, SLC25A13-PE5 comprises the sequence of SEQ ID NO: 29.

In one embodiment, the method as described herein comprises providing an SSO having a binding site that lies within SLC25A13-PE5.

In one embodiment, the method as described herein comprises providing an SSO having a binding site that overlaps with the acceptor splice site of SLC25A13-PE5 and with or a part of SLC25A13-PES.

In one embodiment, the method as described herein comprises providing an SSO having a binding site that overlaps with or a part of SLC25A13-PE5 and with the donor splice site of SLC25A13-PE5.

In one embodiment, the method as described herein comprises providing an SSO having a sequence selected from the group consisting of SEQ ID NOs 1 to 12.

In one embodiment, the method as described herein comprises providing an SSO having a sequence selected from the group consisting of SEQ ID NOs 13 to 27.

In one aspect, there is provided a splice-switching oligonucleotide (SSO) that binds to a site within a target region present on a pre-mRNA transcript of the SLC25A13 gene, the target region having at least 95% sequence identity to SEQ ID NO: 28, and wherein binding of the SSO induces the exclusion of SLC25A13-PE5 from a mature mRNA transcript of the SLC25A13 gene.

In one embodiment, the SSO as described herein has a binding site that lies within SLC25A13-PE5 and wherein SLC25A13-PE5 comprises the sequence of SEQ ID NO: 29.

In one embodiment, the SSO as described herein has a binding site that overlaps with the acceptor splice site of SLC25A13-PE5 and with or a part of SLC25A13-PE5, and wherein SLC25A13-PE5 comprises the sequence of SEQ ID NO: 29.

In one embodiment, the SSO as described herein has a binding site that overlaps with or a part of SLC25A13-PE5 and with the donor splice site of SLC25A13-PE5, and wherein SLC25A13-PE5 comprises the sequence of SEQ ID NO: 29.

In one embodiment, the SSO as described herein comprises a sequence selected from the group consisting of SEQ ID NOs 1 to 12.

In one embodiment, the SSO as described herein comprises a sequence selected from the group consisting of SEQ ID NOs 13 to 27.

In one aspect, there is provided an SSO as described herein for use in treating citrin deficiency.

In one aspect, there is provided a use of an SSO as described herein in the manufacture of a medicament for treating citrin deficiency.

In one aspect, there is provided a method of treating citrin deficiency comprising administering to a subject a composition comprising an SSO as described herein.

In one embodiment, the SSO as described herein is between 15 and 40 nucleotides in length.

In one embodiment, at least one of the nucleotides of the SSO is chemically modified and the chemical modification is 2′-O-methyl RNA modification, 2′-O-methoxyethyl RNA modification, locked nucleic acid substitution, or phosphorothioate linkage.

In one embodiment, the SSO as described herein comprises phosphorothioate linkages between all nucleotides of the SSO.

In one embodiment, each nucleotide of the SSO as described herein comprises either a 2′-O-methyl RNA modification, a 2′-O-methoxyethyl RNA modification or a locked nucleic acid substitution.

In one aspect, there is provided a pharmaceutical composition comprising (a) a therapeutically effective amount of an SSO as described herein and (b) one or more pharmaceutically acceptable carriers and/or diluents.

### BRIEF DESCRIPTION OF THE DRAWINGS

In order that the present invention may be fully understood and readily put into practical effect, there shall now be described by way of non-limitative examples only preferred embodiments of the present invention, the description being with reference to the accompanying illustrative figures.

In the Figures:

FIG. 1 is a schematic of a minigene system that harbours the specific genomic mutation, c.469-2922G>T of the SLC25A13 gene (accession number NM_001160210.2). The minigene includes the full sequence of exon 5, the first 2,000 bases (from exon 5 donor splice site) and last 4,923 bases of intron 5 (or 4,923 bases before exon 6 acceptor site), and the complete sequence of exon 6. The relative loci of the G-to-T substitution is demarcated in the figure.

FIG. 2 shows that CRISPR knock-in and minigene recapitulates the generation of SLC25A13-PE5 (c.469-2909 to c.469-2825 with reference to NM_001160210.2). FIG. 2A is a gel electrophoresis image of PCR products from mRNA extracted from hepatocytes differentiated from human embryonic stem cells (i.e. induced hepatocytes, or iHeps) containing CRISPR knock-in of the c.469-2922G>T mutation. HET is a heterozygous clone, and HOM1 and HOM2 are two separate homozygous clones containing the c.469-2922G>T mutation. CAPN10 serves as a loading control. The data shows that CRISPR knock-in of the c.469-2922G>T mutation recapitulates the retention of the pseudoexon SLC25A13-PE5 in endogenous SLC25A13 mature mRNA. FIG. 2B is an immunoblot image confirming the loss of full length citrin protein in mutant iHeps as a result of the incorporation of SLC25A13-PE5 into SLC25A13 mature transcripts. FIG. 2C is a graph indicating loss of ureagenic potential and FIG. 2D is a graph revealing loss of ammonia clearing capability in mutant iHeps. FIG. 2E is a graph showing that hepatocyte differentiation markers ALB and ASGR1 are not significantly different in iHeps harbouring wildtype or mutant (heterozygous or homozygous for c.469-2922G>T) SLC25A13, suggesting that loss of citrin does not impact hepatocyte differentiation, and that the loss of ureagenic potential and ammonia clearing capability are not due to a difference in hepatocyte differentiation. FIG. 2F is a gel electrophoresis image showing PCR products from three cell lines (HEK293T, Huh7 and HepG2) transfected with either pCIT2 (WT minigene construct) or pCIT2mut (mutant minigene construct). The minigene containing the same mutation recapitulates the retention of the pseudoexon SLC25A13-PE5 in mature messenger RNA (mRNA). All three cell lines showed correct splicing of exon 5 to exon 6 when transfected with pCIT2, and all three cell lines show incorporation of the 85 bp pseudoexon when transfected with pCIT2mut. FIG. 2G is a sample sequencing data showing the incorporation of the pseudoexon SLC25A13-PE5 in mature mRNA from cells transfected with pCIT2mut.

FIG. 3 is a capillary electrophoresis image from screening of SSOs against SLC25A13-PE. Huh7 were co-transfected with 500 ng of pCIT2mut and 25 nM of SSOs, after which PCR was performed on complementary DNA (cDNA) generated from RNA extracted from the transfected cells. The data reveals efficacy of the SSOs in mediating splice exclusion of SLC25A13-PE5. Every ribose sugar moiety in a SSO is modified with 2′-O-methyl that is linked via phosphorothioate (PS) backbone.

FIG. 4 is a graph showing SSO concentration response curves of selected SSOs from the screen in FIG. 3. Huh7 were co-transfected with 500 ng of pCIT2mut and SSOs at the indicated amounts, after which PCR was performed on cDNA generated from RNA extracted from the transfected cells. Capillary electrophoresis was used on PCR products to quantify the amount of products with the pseudoexon and without the pseudoexon. Efficacy is reflected as the percentage of PCR products without the pseudoexon over the total amount of PCR products (% splicing correction).

FIG. 5 shows the result of screening of shortened sequences and mixmers. FIG. 5A is a graph showing splicing correction efficacy of SSOs #2032, #2033 and #2034 modified with 2′O-methyl (20M) or 2′-O-methoxyethyl (2MOE), all of which are shortened from #2008, when Huh7 cells stably expressing the mutant minigene were treated at 200 nM by free uptake in calcium-enriched medium (CEM). #2008 serves as positive control. FIG. 5B is a graph showing the screening of mixmers derived from the sequence of #2034 when transfected at 10 nM into HepG2 stably expressing the mutant minigene. In both sets of experiments, PCR was performed on cDNA generated from RNA extracted from the transfected cells. Capillary electrophoresis was used on PCR products to quantify the amount of products with the pseudoexon and without the pseudoexon. Efficacy is reflected as the percentage of PCR products without the pseudoexon over the total amount of PCR products (% splicing correction). Every ribose sugar moiety in a mixmer is either modified with 2′-O-methyl or 2′-O-methoxyethyl substituted with a locked nucleic acid (2OML or 2MOL, respectively) that is linked via phosphorothioate backbone.

FIG. 6 shows the results of screening of shortened mixmers used in FIG. 5 by free uptake in calcium-enriched medium (CEM). FIG. 6A is a graph showing screening of mixmers by free uptake under CEM on Huh7 or HepG2 cells stably expressing the mutant minigene when treated with 20 nM or 200 nM of SSOs, respectively. The use of CEM stimulates in vitro uptake by cells and better reflects the in vivo efficacy when compared to transfection. The data shows that the shortened mixmers were able to modulate splice-out of SLC25A13-PE5 in the absence of a transfection agent. FIG. 6B is a graph showing the free uptake dose response curve of selected SSOs, revealing good efficacy of #2034.5 and #2034.15 with EC₅₀ in the nanomolar range in Huh7 cells stably expressing the mutant minigene. In fact, EC₅₀ of the two mixmers are about 10 times lower than their parent SSO (#2008) and the singly 2′-O-methyl modified #2034. FIG. 6C is a graph of MTT assay whereby Huh7 cells stably expressing the mutant minigene were treated by free uptake under CEM with different concentrations of SSOs for 72 hours; SSOs with a “C” prefix are additional negative controls whose sequence is full complementary with identical chemical modifications as the corresponding SSO lead #2034. As each “C”-prefixed SSO was chemically modified in the exact pattern as its corresponding mixmer, it serves as a negative control in the context of chemical modifications. The data suggests a lack of toxicity caused by the sequence and chemistry combinations. LNA32 with the sequence EAAaggaaacacaEAT (SEQ ID NO: 46) and LNA41 with the sequence EAEattccttgctETG (SEQ ID NO: 47) are gapmers with full PS backbone linkage containing natural DNA bases (in small letters) flanked by three LNAs (in capital letters) on both ends that were previously shown to be respectively non-toxic and acutely hepatotoxic in vivo in mouse studies. Therefore, LNA32 serves as a negative control and LNA41 serves as a positive control. In SEQ ID NOs 46 and 47, capital letters represent LNA, small letters represent DNA, and “E” represents 5-methylcytosine.

FIG. 7 shows results from asialoglycoprotein receptor-mediated functional uptake of SSOs (in the absence of CEM and a transfection agent) in iHeps homozygous for SLC25A13-PE5 with 4 μM of GaINAc*3 (GN*3)-conjugated non-targeting control (NC2 g1.1), 2034.5 (2034.5g1.1) or 2034.15 (2034.15g1.1) in either 2′-O-methyl+LNA (2OML) or 2′-O-methoxyethyl+LNA (2MOL) chemistry combinations. Three GaINAc molecules, GN*3, were chemically bonded in a trivalent configuration to the 5′ of each SSO, which allow for receptor-mediated uptake of the SSOs through the asialoglycoprotein receptor (ASGR1/2) expressed specifically on hepatocytes (FIG. 2E). FIG. 7A reveals that the GalNAc-conjugated SSOs 2034.5 and 2034.15, but not NC2, can modulate splice-out of SLC25A13-PE5 from endogenous SLC25A13 transcripts. FIG. 7B displays expression of total SLC25A13 transcripts in mutant iHeps, reflected as relative expression upon normalization to WT iHeps treated with the respective NC2g1.1 non-targeting control (2OM for 2OML SSOs or 2MOE for 2MOL SSOs). The data suggests that the splicing correction by the GaINAc-conjugated SSOs rescues SLC25A13 transcript levels, which were degraded by nonsense-mediated decay in mutant iHeps. FIG. 7C shows rescue of ureagenesis and FIG. 7D highlights rescue of ammonia clearance when mutant iHeps were treated with GaINAc-conjugated SSOs. Data shown are of mutant iHeps normalized to WT iHeps treated with the respective NC2g1.1 non-targeting control. FIG. 7E is a graph of expression of acute toxicity markers CDKN1A, BAX and PUMA in mutant iHeps treated with SSOs, shown relative to WT iHeps treated with NC2g1.1. The data hints at lack of acute toxicity in treated mutant iHeps with no significant increase in expression of acute toxicity markers CDKN1A, BAX and PUMA when compared to WT iHeps treated with NC2g1.1. FIG. 7F shows an immunoblot image whereby expression of full length citrin protein is rescued in iHeps homozygous for SLC25A13-PE5 (HOM1) treated with GaINAc-conjugated #2034.15 2OML (GN*3-2034.15 2OML). This would corroborate with the functional data, suggesting that the rescue in the functional assays (FIGS. 7C and 7D) are likely due to rescue in the protein level, and thus protein activity.

### DETAILED DESCRIPTION OF THE PRESENT INVENTION

In an aspect of the invention, there is provided a method of exon-skipping comprising providing a splice-switching oligonucleotide (SSO) that binds to a site within a target region present on a pre-mRNA transcript of the SLC25A13 gene, wherein the binding of the SSO induces the exclusion of SLC25A13-PE5 from a mature mRNA transcript of the SLC25A13 gene.

By “oligonucleotide”, it is meant to refer to any polynucleotide. A “polynucleotide” is an oligomer comprised of nucleotides. A polynucleotide may be comprised of DNA, RNA modified forms thereof, or a combination thereof. The term “nucleotide” or its plural as used herein is interchangeable with modified forms as discussed herein and otherwise known in the art. In certain instances, the art uses the term “nucleobase” which embraces naturally occurring nucleotides as well as modifications of nucleotides that can be polymerized. Thus, nucleotide or nucleobase means the naturally occurring nucleobases adenine (A), guanine (G), cytosine (C), thymine (T) and uracil (U) as well as non-naturally occurring nucleobases such as xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C[3]-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et ah, U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al), in Chapter 15 by Sanghvi, in Antisense Research and Application, Ed. S. T. Crooke and B. Lebleu, CRC Press, 1993, in Englisch et ah, 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which is hereby incorporated by reference in its entirety). In various embodiments, polynucleotides also include one or more “nucleosidic bases” or “base units” which include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles {e.g., 5-nitroindole), and optionally substituted hypoxanthine. Other desirable universal bases include pyrrole, and diazole or triazole derivatives, including those universal bases known in the art.

Polynucleotides may also include modified nucleobases. A “modified base” is understood in the art to be one that can pair with a natural base (e.g., adenine, guanine, cytosine, uracil, and/or thymine) and/or can pair with a non-naturally occurring base. Exemplary modified bases are described in EP 1 072 679 and WO 97/12896, the disclosures of which are incorporated herein by reference. Modified nucleobases include, without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(IH-pyrimido[5,4-b][1,4]benzoxazin-2(3H)-one), phenothiazine cytidine (IH-pyrimido[5,4-b][I,4]benzothiazin-2(3H)-one), G-clamps such as a substituted phenoxazine cytidine (e.g. 9-(2-aminoethoxy)-H-pyrimido[5,4-b][I,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al, 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity of the polynucleotide and include 5-substituted pyrimidines, 6-azapyrimidines and N-2, N-6 and 0-6 substituted purines, including 2-aminopropyladenine, 5-propynyluracil and 5-propynylcytosine, 5-methylcytosine substitutions have been shown to increase nucleic acid duplex stability by 0.6-1.2.deg.C and are, in certain embodiments, combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594, 121, 5,596,091; 5,614,617; 5,645,985; 5,830,653; 5,763,588; 6,005,096; 5,750,692 and 5,681,941, the disclosures of which are incorporated herein by reference.

As used herein, the term “splice switching oligonucleotides” (SSOs) or “splice switching oligomers” is meant to include synthetic antisense nucleic acids that base-pair with a pre-mRNA and disrupt the splicing process by sterically blocking the RNA-RNA base-pairing or protein-RNA binding interactions that occur between components of the splicing machinery and the pre-mRNA. The SSOs may also be known as “antisense nucleotides”, “steric blockers” or “steric hindrance antisense nucleotides” which can modulate splicing. SSOs may modulate splicing via steric blocking. In some embodiments, SSOs may be mixmers. The term “mixmer” includes an oligomer on which different types of chemical modifications are applied on its sugar moieties, or on its backbone linkages, or both. Examples of chemical modifications include phosphorothioate linkages, 2′-O-methyl RNA modifications, 2′-O-methoxyethyl RNA modifications and locked nucleic acid substitutions. The terms “phosphorothioate bond” and “phosphorothioate linkage” are used interchangeably. The chemical modifications may increase the efficacy, selectivity and stability while manifesting superior toxicity profile of SSOs.

The term “splicing” refers to an RNA processing mechanism in which a pre-mRNA is made into a mature mRNA. During splicing, introns are removed and exons are connected. Splicing is catalysed by the spliceosome complex. As used herein, the term “alternative splicing” is meant to include a process by which a gene can encode for multiple mRNA and protein products by differentially selecting which exons are to be included in a mature mRNA transcript. For example, alternative splicing can take the form of one or more skipped exons, variable position of intron splicing, or intron retention.

As used herein, the term “intron” refers to a segment of non-coding nucleic acid sequence that is transcribed and is present in the pre-mRNA but is excised by the splicing machinery and therefore not present in the mature mRNA transcript.

As used herein, the term “exon” refers to a segment of a nucleic acid sequence that is transcribed into mRNA and that is present in mature mRNA after splicing. The term “exon skipping” is meant to include the process by which an entire exon, or a portion thereof, is removed from a given pre-mRNA and is thereby excluded from being present in the mature mRNA. For example, the portion of the protein that is otherwise encoded by the skipped exon is not present in the expressed form of the protein.

In one embodiment, the target region has at least 95% sequence identity to SEQ ID NO: 28. In various embodiments, the target region may include a variant sequence of SEQ ID NO: 28. The target region may comprise, consist or consist substantially or essentially of a sequence having at least 95%, 96%, 97%, 98%, 99% ot 100% sequence identity thereto.

As used herein, the term “splice site” is meant to include specific nucleic acid sequences that can be recognized by the splicing machinery as being suitable for excision and/or ligation with the corresponding splice site. The splice site defines the precise exon-intron boundary that allows the excision of introns present in pre-mRNA transcripts. As used herein, the term “5′ splice site” (also known as donor splice site) refers to a nucleic acid sequence surrounding the exon-intron boundary at the 5′ end of an intron that marks the start of the intron and its boundary with the preceding exon sequence. The term “3′ splice site” (also known as acceptor splice site) as used herein refers to a nucleic acid sequence surrounding the intron-exon boundary at the 3′ end of an intron that marks the end of the intron and its boundary with the following exon sequence.

As used herein, the term “pre-mRNA” or “precursor mRNA” refers to a strand of messenger ribonucleic acid (mRNA), synthesized from a DNA template by transcription. Pre-mRNA is composed of exons, introns and untranslated sequences (before the first and after the last exons respectively). Generally, eukaryotic pre-mRNA exists only briefly before it is fully processed into mature mRNA.

The term “binding” as used in the context of an SSO is meant to include the hybridization of the SSO to a site within a target region on a pre-mRNA transcript. The term “hybridize” or “hybridization” may include the binding of a single-stranded nucleic acid or a locally single-stranded region of a double-stranded nucleic acid to another single-stranded nucleic acid or a locally single-stranded region of a double-stranded nucleic acid having a complementary sequence through the pairing of complementary nucleic acids. It is generally known to a person skilled in the art that binding or hybridization of one sequence to another does not require total complementarity of the sequences. For example, the sequence of the SSO may be completely complementary or partially complementary to the target region to which it binds.

The term “site” refers to a location that the SSO is substantially or fully complementary to. The SSO may bind to this site. In the context of “a site within a target region on a pre-mRNA transcript”, the site lies within the target region, and the target region forms a part of the pre-mRNA transcript.

By “variant” in the relevant gene, it is meant to include any variation or alteration in the sequences of said gene, such that the sequence differs from what is found naturally or in most people. Similarly, a “non-variant” may include any sequence of the gene that may be considered “wild-type”, i.e. a sequence that is deemed normal or typical for said gene. As such, a “variant” of the gene means any one or more alteration(s), i.e. a substitution, duplication, inversion, insertion, and/or deletion, at one or more (several) positions, of the polynucleotide of the gene. A substitution may include a replacement of one or more nucleotide(s) occupying a position with one or more different nucleotide(s); a deletion means removal of one or more nucleotide(s) occupying a position; and an insertion means adding one or more nucleotide(s) immediately adjacent to a nucleotide occupying a position. The term “variant” may also refer to any variation or alteration in the sequence of a gene that results in the loss of wild-type protein expression and/or function, or gain-of-function.

In one embodiment, the pre-mRNA transcript of the SLC25A13 gene is a pre-mRNA transcript of a variant of the SLC25A13 gene. The variant of the SLC25A13 gene may comprise a c.469-2922G>T mutation. In one embodiment, the binding site of the SSO as described herein resides within a target region of 5′-CCUCCCAUUGUUCAAUAGCUCACGAUUUGUUCAUUCAUUUGGUUUUACAGAAUACUU UUCACUGAUGAGAAUGCCUGUCAUUUAUUGAGCACCUACUAUACAUCUAAAGCAUUC UGCUGAGCUGCAUGUAUAAAUGUAAGUAGAUGCUUACAGGACUUCAAAAGGUUAUAC UGUCUUUUCCUUGG-3′ (SEQ ID NO: 28). The cDNA sequence that encodes for SEQ ID NO: 28 is 5′-CCTCCCATTGTTCAATAGCTCACGATTTGTTCATTCATTTGGTTTTACAGAATACTTTTCA CTGATGAGAATGCCTGTCATTTATTGAGCACCTACTATACATCTAAAGCATTCTGCTGAG CTGCATGTATAAATGTAAGTAGATGCTTACAGGACTTCAAAAGGTTATACTGTCTTTTCC TTGG-3′ (SEQ ID NO: 44). It would be generally understood that a skilled person given SEQ ID NO: 44 would know how to derive the RNA sequence of the target region (i.e. SEQ ID NO: 28). SEQ ID NO:28 comprises the sequence of SLC25A13-PE5 (SEQ ID NO: 29) as well as the sequence of partial introns flanking SLC25A13-PE5. The sequence of partial introns flanking SLC25A13-PE5 include the acceptor and donor splice sites of SLC25A13-PE5. The c.469-2922G>T mutation in SEQ ID NO: 44 and the corresponding G>U mutation in SEQ ID NO: 28 are shown in bold and underline above. In one embodiment, the binding site of the SSO may overlap with the SLC25A13-PE5 acceptor splice site and with or a part of SLC25A13-PE5. In another embodiment, the entire binding site of the SSO may lie within SLC25A13-PE5. In yet another embodiment, the binding site of the SSO may overlap with or a part of SLC25A13-PE5 and the SLC25A13-PE5 donor splice site.

In one embodiment, SLC25A13-PE5 comprises the sequence of SEQ ID NO: 29.

The term “pseudoexon” refers to a potential exon, containing adequate 5′ and 3′ splice sites, that is not normally spliced into mature mRNA by the splicing machinery. The inclusion of a pseudoexon in a mature mRNA, for example due to a mutation that either creates/activates or eliminates/diminishes a splicing motif or splice site, or dysregulation of the splicing machinery stemming from the absence or overproduction of one or more components of the spliceosome complex or specific RNA binding protein(s) acting as splicing enhancers or splicing silencers, may cause a shift in the codon reading frame, an in-frame premature stop codon, or addition of novel amino acid residues, resulting in a loss of expression/function of the protein. For clarity, the genetic mutation(s) that effects the creation of a pseudoexon need not reside within the pseudoexon.

In one embodiment, the term “pseudoexon” as used herein refers to SLC25A13-PE5 with the RNA sequence 5′-AAUACUUUUCACUGAUGAGAAUGCCUGUCAUUUAUUGAGCACCUACUAUACAUCUAA AGCAUUCUGCUGAGCUGCAUGUAUAAAU-3′ (SEQ ID NO: 29). The cDNA sequence that encodes for SLC25A13-PE5 is 5′-AATACTTTTCACTGATGAGAATGCCTGTCATTTATTGAGCACCTACTATACATCTAAAGC ATTCTGCTGAGCTGCATGTATAAAT-3′ (SEQ ID NO: 45). It would be generally understood that a skilled person given SEQ ID NO: 45 would know how to derive the RNA sequence of SLC25A13-PE5 (i.e. SEQ ID NO: 29). The terms “SLC25A13-PE5”, “SLC25A13-PE”, “PE” and “Exon 5*” may be used interchangeably.

In one embodiment, the method as described herein comprises providing an SSO having a binding site that lies within SLC25A13-PE5.

In one embodiment, the method as described herein comprises providing an SSO having a binding site that overlaps with the acceptor splice site of SLC25A13-PE5 and with or a part of SLC25A13-PE5.

In one embodiment, the method as described herein comprises providing an SSO having a binding site that overlaps with or a part of SLC25A13-PE5 and with the donor splice site of SLC25A13-PE5.

In various embodiments, the method as described herein comprises providing an SSO having a sequence selected from the group consisting of SEQ ID NOs 1 to 12.

In various embodiments, the method as described herein comprises providing an SSO having a sequence selected from the group consisting of SEQ ID NOs 13 to 27.

In one aspect of the present invention, there is provided a splice-switching oligonucleotide (SSO) that binds to a site within a target region present on a pre-mRNA transcript of the SLC25A13 gene, the target region having at least 95% sequence identity to SEQ ID NO: 28, and wherein binding of the SSO induces the exclusion of SLC25A13-PE5 from a mature mRNA transcript of the SLC25A13 gene.

In various embodiments, the SSO has a binding site that lies within SLC25A13-PE5 and wherein SLC25A13-PE5 comprises the sequence of SEQ ID NO: 29.

In various embodiments, the SSO has a binding site that overlaps with the acceptor splice site of SLC25A13-PE5 and with or a part of SLC25A13-PE5, and wherein SLC25A13-PE5 comprises the sequence of SEQ ID NO: 29.

In various embodiments, the SSO has a binding site that overlaps with or a part of SLC25A13-PE5 and with the donor splice site of SLC25A13-PE5, and wherein SLC25A13-PE5 comprises the sequence of SEQ ID NO: 29.

In various embodiments, the SSO of the invention comprises a sequence selected from the group consisting of SEQ ID NOs 1 to 12. In various embodiments, the SSO comprises a sequence selected from the group consisting of SEQ ID NOs 13 to 27. The SSO may be between 15 and 40 nucleotides in length.

Besides c.469-2922G>T, other mutations may be able to cause the generation of SLC25A13-PE5. Examples of the other mutations that may result in the inclusion of SLC25A13-PE5 include c.469-2922G>C, c.469-2922G>A, c.469-2923A>T, c.469-2923A>G, c.469-2923A>C, c.469-2923_469-2920del, c.469-2922_469-2921del, c.469-2923_469-2921del, c.469-2924_469-2921del, c.469-2922del, c.469-2923_469-2922del, c.469-2924_469-2922del, c.469-2924_469-2921del, c.469-2923_469-2922insT, c.469-2923_469-2922insC, c.469-2923del, c.469-2924_469-2923del and c.469-2925_469-2923del. It would be generally appreciated by the skilled person that binding or hybridization of one sequence to another does not require total complementarity of the sequences. Therefore, SSOs with a sequence that is not completely complementary to the target region of SEQ ID NO: 28 would also be able to bind to the target region. For example, SSOs complementary to a pre-mRNA resulting from a mutation selected from the group consisting of c.469-2922G>C, c.469-2922G>A, c.469-2923A>T, c.469-2923A>G, c.469-2923A>C, c.469-2923_469-2920del, c.469-2922_469-2921del, c.469-2923_469-2921del, c.469-2924_469-2921del, c.469-2922del, c.469-2923_469-2922del, c.469-2924_469-2922del, c.469-2924_469-2921del, c.469-2923_469-2922insT, c.469-2923_469-2922insC, c.469-2923del, c.469-2924_469-2923del and c.469-2925_469-2923del would also be able to bind to SEQ ID NO: 28.

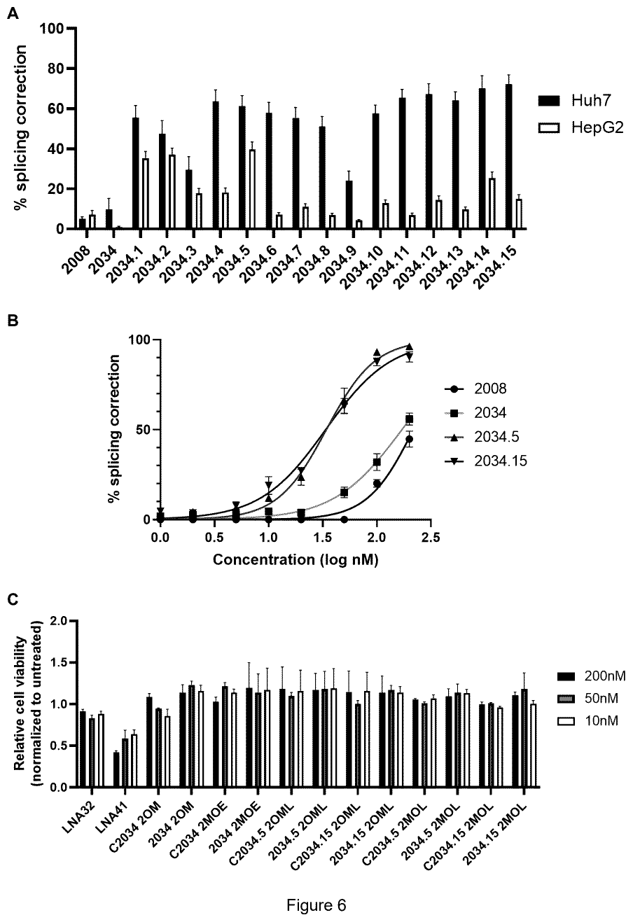

Advantageously, the SSOs of the present embodiments are able to bind to the respective binding sites on the target region competitively due to favourable binding thermodynamics and extent of co-transcriptional locally single-stranded binding site on the target region identified. Selection of the target region involves considering the presence of RNA-binding protein motifs on the target region. The SSOs of the present embodiments are able to induce the desired splicing modulation by competitive binding to target sites that encompass or overlap sequence motifs used by the appropriate RNA-binding protein(s), snRNPs (small nuclear ribonucleoproteins), or both. Advantageously, shortened SSOs reduce propensity of immune response and may have superior uptake kinetics by cells, as shown in FIG. 6.

In various embodiments, at least one of the nucleotides of the SSO is chemically modified and wherein the chemical modification is 2′-O-methyl RNA modification, 2′-O-methoxyethyl RNA modification, locked nucleic acid substitution, or phosphorothioate linkage. The term “locked nucleic acid” (LNA) generally refers to a modified RNA nucleotide where the ribose ring is “locked” with a methylene bridge connecting the 2′-O atom with the 4′-C atom.

Modified polynucleotides are contemplated for use wherein both one or more sugar and/or one or more internucleotide linkage of the nucleotide units in the polynucleotide is replaced with “non-naturally occurring” sugars (i.e., sugars other than ribose or deoxyribose) or internucleotide linkages, respectively. In one embodiment, this embodiment contemplates a peptide nucleic acid (PNA). In PNA compounds, the sugar-backbone of a polynucleotide is replaced with an amide-containing (e.g., peptide bonds between N-(2-aminoethyl)-glycine units) backbone. See, for example U.S. Pat. Nos. 5,539,082; 5,714,331; and 5,719,262, and Nielsen et ah, Science, 1991, 254, 1497-1500, the disclosures of which are herein incorporated by reference. Modified polynucleotides may also contain one or more substituted sugar groups. In one embodiment, a modification of the sugar includes Locked Nucleic Acids (LNAs) in which the 2′-hydroxyl group is linked to the 3′ or 4′ carbon atom of the sugar ring, thereby forming a bicyclic sugar group. The linkage is in certain embodiments a methylene (—CH_{[2]}—)_{[n]} group bridging the 2′ oxygen atom and the 4′ carbon atom wherein n is 1 or 2. LNAs and preparation thereof are described in WO 98/39352 and WO 99/14226, the disclosures of which are incorporated herein by reference. In the present invention, preferably, the antisense oligonucleotide comprises a modified polynucleotide backbone. The modified polynucleotide backbone may comprise a modified moiety substituted for the sugar of at least one of the polynucleotides. The modified moiety may be selected from the group comprising of phosphorodiamidate morpholino oligomer (PMO), peptide-conjugated phosphorodiamidate morpholino oligomer (PPMO), and non-peptide dendrimeric octaguanidine moiety-tagged morpholino oligomer.

In various embodiments, the modified polynucleotide backbone comprises at least one modified internucleotide linkage. The modified internucleotide linkage comprises a modified phosphate. More preferably, the modified phosphate is any one selected from the group comprising of a non-bridging oxygen atom substituting a sulfur atom, a phosphonate, a phosphorothioate, a phosphodiester, a phosphoromorpholidate, a phosphoropiperazidate and a phosphoroamidate.

In various embodiments of the invention, the SSO comprises a backbone selected from the group comprising of ribonucleic acid, deoxyribonucleic acid, DNA phosphorothioate, RNA phosphorothioate, 2′-O-methyl-oligoribonucleotide and 2′-O-methyl-oligodeoxyribonucleotide, 2′-O-hydrocarbyl ribonucleic acid, 2′-O-hydrocarbyl DNA, 2′-O-hydrocarbyl RNA phosphorothioate, 2′-O-hydrocarbyl DNA phosphorothioate, 2′-F-phosphorothioate, 2′-F-phosphodiester, 2′-methoxyethyl phosphorothioate, 2-methoxyethyl phosphodiester, deoxy methylene(16ore16lamino) (deoxy MMI), 2′-O-hydrocarby MMI, deoxy-methylphos-phonate, 2′-O-hydrocarbyl methylphosphonate, morpholino, 4′-thio DNA, 4′-thio RNA, peptide nucleic acid, 3′-amidate, deoxy 3′-amidate, 2′-O-hydrocarbyl 3′-amidate, locked nucleic acid, cyclohexane nucleic acid, tricycle-DNA, 2′-fluoro-arabino nucleic acid. N3′-P5′ phosphoroamidate, carbamate linked, phosphotriester linked, a nylon backbone modification and mixtures of the aforementioned backbones.

In various embodiments, the oligonucleotide is chemically linked to one or more conjugates that enhance the activity, cellular distribution, or cellular uptake of the SSO.

In various embodiments, the SSO comprises phosphorothioate linkages between all nucleotides of the SSO.

In various embodiments, each nucleotide of the SSO comprises either a 2′-O-methyl RNA modification, a 2′-O-methoxyethyl RNA modification or a locked nucleic acid substitution.

In another aspect of the invention, there is provided the SSOs of this invention for use in medicine or in treating citrin deficiency. As used herein, the term “treat” or “treating” in the context of treating a disease such as citrin deficiency is meant to include improving clinical outcomes of patients having the disease. This includes improving the survival rates of patients having the disease. The term “treat” or “treatment” may refer to prophylactic and/or therapeutic treatment.

In one aspect, there is provided a use of an SSO as described herein the manufacture of a medicament for treating citrin deficiency. In another aspect, there is provided a method of treating citrin deficiency comprising administering to a subject a composition comprising an SSO as described herein. These SSOs may be used in compositions that can be used for treatment, e.g. as a pharmaceutical composition comprising the SSO of the invention and a pharmaceutically acceptable carrier. The composition is suitable for parenteral administration either naked or complexed with a delivery agent to a patient. The carrier is selected from the group consisting of a nanoparticle, such as a polymeric nanoparticle; a liposome, such as pH-sensitive liposome, an antibody conjugated liposome; a viral vector, a cationic lipid, a polymer, a UsnRNA, such as U7 snRNA and a cell penetrating peptide. The SSO is administered orally, or rectal, or transmucosal, or intestinal, or intramuscular, or subcutaneous, or intramedullary, or intrathecal, or direct intraventricular, or intravenous, or intravitreal, or intraperitoneal, or intranasal, or intraocular.

A pharmaceutically acceptable carrier refers, generally, to materials that are suitable for administration to a subject wherein the carrier is not biologically harmful, or otherwise, causes undesirable effects. Such carriers are typically inert ingredients of a medicament. Typically a carrier is administered to a subject along with an active ingredient without causing any undesirable biological effects or interacting in a deleterious manner with any of the other components of a pharmaceutical composition in which it is contained. Suitable pharmaceutical carriers are described in Martin, Remington's Pharmaceutical Sciences, 18^{th} Ed., Mack Publishing Co., Easton, Pa., (1990), incorporated by reference herein in its entirety.

In a more specific form of the disclosure there are provided pharmaceutical compositions comprising therapeutically effective amounts of an SSO together with pharmaceutically acceptable diluents, preservatives, solubilizers, emulsifiers, adjuvants and/or carriers. Such compositions include diluents of various buffer content (e.g., phosphate, Tris-HCl, acetate), pH and ionic strength and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The material may be incorporated into particulate preparations of polymeric compounds such as, for example and without limitation, polylactic acid or polyglycolic acid, or into liposomes. Hylauronic acid may also be used. Such compositions may influence the physical state, stability, rate of in vivo release, and rate of in vivo clearance of the disclosed compositions. The compositions may be prepared in liquid form, or may be in dried powder, such as lyophilized form.

It will be appreciated that pharmaceutical compositions provided according to the disclosure may be administered by any means known in the art. Preferably, the pharmaceutical compositions for administration are administered by injection, orally, or by the pulmonary, or nasal route. The antisense polynucleotides are, in various embodiments, delivered by intravenous, intra-arterial, intraperitoneal, intramuscular, or subcutaneous routes of administration.

The oligonucleotides of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

For polynucleotides, preferred examples of pharmaceutically acceptable salts include, but are not limited to, (a) salts formed with cations such as sodium, potassium, ammonium, magnesium, calcium, polyamines such as spermine and spermidine; (b) acid addition salts formed with inorganic acids, for example hydrochloric acid, hydrobromic acid, sulfuric acid, phosphoric acid, nitric acid; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid; and (d) salts formed from elemental anions such as chlorine, bromine, and iodine. The pharmaceutical compositions of the disclosure may be administered in a number of ways depending upon whether local or systemic treatment is desired and upon the area to be treated. Administration may be topical (including ophthalmic and to mucous membranes including rectal delivery), pulmonary, e.g., by inhalation of powders or aerosols, (including by nebulizer, intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration.

The pharmaceutical formulations of the disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly bringing into association the active ingredients with liquid carriers or finely divided solid carriers or both, and then, if necessary, shaping the product.

Combination therapy with an additional therapeutic agent may also be contemplated by the disclosure. The term “combination” or “combination therapy” as used throughout the specification, is meant to encompass the administration of the referred therapeutic agents to a subject suffering from a disease, disorder or pathological condition, in the same or separate pharmaceutical formulations, and at the same time or at different times. If the therapeutic agents are administered at different times they should be administered sufficiently close in time to provide for the potentiating or synergistic response to occur. In such instances, it is contemplated that one would typically administer both therapeutic agents within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In other situations, it might be desirable to reduce the time between administration, administering both therapeutic agents within seconds or minutes to hours, preferably within about 6 hours from each other, more preferably within about 1 or 3 hours.

The term “therapeutically effective amount” refers to the amount of the SSO that is required to confer the intended therapeutic effect in the subject, which amount will vary depending on the route of administration, status of disease, age, gender, body weight, and possible inclusion of other therapeutics or excipients. The method and uses of the invention are for a patient in need thereof. The compositions and methods of this invention are for a subject or patient in need thereof. The term “patient in need thereof” refers to a person who has or is suspected of having or developing citrin deficiency, as well as a person who is predisposed to but yet to develop citrin deficiency.

To practice the methods of this invention, the SSO may be administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parentally acceptable diluents or solvent for example, as a solution in 1,3-butanediol. Among the acceptable vehicles and solvents that can be employed are mannitol, water, Ringer's Solution and isotonic sodium chloride solution. In addition, sterile, fixed oils are conventionally employed as a solvent or suspending medium (eg. Synthetic mono- or dyglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluents or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavouring, or colouring agents can be added. A nasal aerosol or inhalation composition can be prepared according to techniques well known in the art of pharmaceutical formulation. A SSO-containing composition can also be administered in the form of suppositories for rectal administration. The carrier in the pharmaceutical composition must be “acceptable” in the sense of being compatible with the active ingredient of the formulation (and preferable, capable of stabilising it) and not deleterious to the subject to be treated. For example, one or more solubilising agents, which form more soluble complexes with the SSOs, or more solubilising agents, can be utilised as pharmaceutical carriers for delivery of the active compounds. Examples of other carriers include colloidal silicon dioxide, magnesium stearate, sodium lauryl sulphate, and D&C Yellow #10

In various embodiments, the method as described herein comprises providing an SSO that is between 15 and 40 nucleotides in length.

In various embodiments, the method as described herein comprises providing an SSO with at least one of the nucleotides of the SSO being chemically modified and wherein the chemical modification is 2′-O-methyl RNA modification, 2′-O-methoxyethyl RNA modification, locked nucleic acid substitution, or phosphorothioate linkage.

In various embodiments, the method as described herein comprises providing an SSO having phosphorothioate linkages between all nucleotides of the SSO.

In various embodiments, the method as described herein comprises providing an SSO with each nucleotide having either a 2′-O-methyl RNA modification, a 2′-O-methoxyethyl RNA modification or a locked nucleic acid substitution.

In one aspect of the present invention, there is provided a pharmaceutical composition comprising (a) a therapeutically effective amount of an SSO as described herein and (b) one or more pharmaceutically acceptable carriers and/or diluents.

The listing or discussion of an apparently prior-published document in this specification should not necessarily be taken as an acknowledgement that the document is part of the state of the art or is common general knowledge.

Any document referred to herein is hereby incorporated by reference in its entirety.

The invention is described in greater detail below.

### Materials and Methods

### Cell Culture

HEK293T, Huh7 and HepG2 cells were maintained in Dulbecco's Modified Eagle Medium (DMEM) with high glucose supplemented with 10% fetal bovine serum and kept in a cell culture incubator set to 37° C. and 5% carbon dioxide. Embryonic stem cells (ES cells) were maintained on ES cell-qualified Matrigel (Corning) in mTeSR Plus medium (STEMCELL Technologies). For hepatocyte differentiation into induced hepatocytes (iHeps), ES cells seeded at 50% confluency were induced in definitive endoderm medium 1 (RPMI 1640 medium supplemented with B-27 supplement, 100 ng/mL Activin A and 3 uM CHIR99021) for two days, definitive endoderm medium 2 (RPMI 1640 medium supplemented with B-27 supplement) for three days, hepatic endoderm medium (definitive endoderm medium 2 with 20 ng/mL BMP4 and 10 ng/mL FGF2) for five days, and Lonza Hepatocyte Culture Medium for fourteen days. CRISPR knock-in of the c.469-2922G>T variant into ES cells was outsourced to the Duke-NUS Stem Cell and Gene Editing (SCAGE) Core Facility.

Co-transfection of 500 ng of minigene plasmid and differing amounts of SSOs into cells were performed using Lipofectamine 3000 reagent (Invitrogen) according to manufacturer's instructions using Opti-MEM (Thermo Fisher). Cells were collected 24 hours post-transfection for RNA analysis and were treated with 50 μg/mL cycloheximide to block NMD about 17 hours prior to cell harvesting.

To establish Huh7 and HepG2 cells stably expressing the minigene, the minigene was sub-cloned into a Piggybac-based transfer vector and co-transfected with a Piggybac transposon-expressing plasmid at a 5:1 ratio for a total of 500 ng using Lipofectamine 3000 as above, after which cells were selected with puromycin at 1 μg/mL for 2 weeks.

Free uptake of SSOs by Huh7 and HepG2 under CEM was performed by treating cells with SSOs in calcium-enriched medium (cell culture media as above supplemented with 9 mM calcium chloride). Cells were collected 72 hours post-treatment for RNA analysis and were treated with 50 μg/mL cycloheximide about 17 hours prior to cell harvesting. For SSO treatment of iHeps, cells were treated with GaINAc-conjugated SSOs in Lonza Hepatocyte Culture Medium for 72 hours. No cycloheximide treatment was performed for SSO-treated iHeps.

### Splicing Assay

RNA was extracted from cells using TRIzol Reagent (Invitrogen) and messenger RNA (mRNA) was converted to complementary DNA (cDNA) using the Maxima First Strand cDNA Synthesis Kit (Thermo Fisher). PCR was performed on cDNA and PCR products were separated using standard gel electrophoresis on agarose gel for qualitative analysis or using capillary electrophoresis on the Qsep100 Bio-Fragment Analyzer (BiOptic Inc) for quantitative analysis. To analyze sequence of PCR products, specific PCR bands were excised and extracted from agarose gel using the QIAquick Gel Extraction Kit (QIAgen) and Sanger sequencing was outsourced to Macrogen Inc.

### Quantitative Real-Time PCR

For quantitation of SLC25A13 and acute toxicity markers CDKN1A, BAX and PUMA mRNA as well as hepatocyte differentiation markers ALB and ASGR1, quantitative real-time PCR was performed on cDNA using PowerUp SYBR Green Master Mix (Applied Biosystems) and detected using the CFX Touch Real-Time PCR Detection System (Bio-Rad). Primers used are: CDKN1A forward 5′-AGCAGAGGAAGACCATGTGGA-3′ (SEQ ID NO: 30), reverse 5′-AATCTGTCATGCTGGTCTGCC-3′ (SEQ ID NO: 31); BAX forward 5′-CCCGAGAGGTCTTTTTCCGAG-3′ (SEQ ID NO: 32), reverse 5′-CCAGCCCATGATGGTTCTGAT-3′ (SEQ ID NO: 33); PUMA forward 5′-GACCTCAACGCACAGTACGAG-3′ (SEQ ID NO: 34), reverse 5′-AGGAGTCCCATGATGAGATTGT-3′ (SEQ ID NO: 35); ALB forward 5′-GTTGCATGAGAAAACGCCAGT-3′ (SEQ ID NO: 36), reverse 5′-GTCGCCTGTTCACCAAGGAT-3′ (SEQ ID NO. 37); ASGR1 forward 5′-GAGACAGAGCTGGACAAG-3′ (SEQ ID NO: 38), reverse 5′-CCCCTTCCCTTAAAATCCT-3′ (SEQ ID NO: 39); SLC25A13 forward 5′-TGGACTGTATAGAGGTCTGTTGC-3′ (SEQ ID NO: 40), reverse 5′-CCCTCACAAAATCGTTCACTGT-3′ (SEQ ID NO: 41); CAPN10 forward 5′-CTTCTGCGACTTGTCTACGCC-3′ (SEQ ID NO: 42), reverse 5′-GTGTGGCACAAATCTCCTGG-3′ (SEQ ID NO: 43). Relative quantification was calculated using the 2^{-ΔΔCt} method with CAPN10 as loading control.

### Immunoblot

Protein was extracted in Radioimmunoprecipitation buffer (50 mM sodium chloride, 50 mM Tris buffer pH6.8, 1 mM ethylenediaminetetraacetic acid, 1% Triton X-100, 0.1% sodium deoxycholate) supplemented with protease and phosphatase inhibitors and quantified using Bradford reagent (Bio-Rad). 30 μg of lysates were denatured in SDS loading dye (1% β-mercaptoethanol, 0.004% bromophenol blue, 6% glycerol, 2% sodium dodecylsulfate, 50 mM Tris buffer pH6.8), then separated by polyacrylamide gels and transferred to PVDF membranes with the Bio-Rad Mini-Protean System, after which membranes were probed with antibodies against anti-SLC25A13 (ab96303, Abcam) and detected using the iBright FL1500 (Invitrogen).

### Urea and Ammonia Assay

After iHeps were treated with GaINAc-conjugated SSOs for 72 hours, media was refreshed with HCM supplemented with 2 mM ammonium chloride and urea was collected for 48 hours. Urea in the media was quantified using QuantiChrom Urea Assay Kit (BioAssay Systems) and signal detected using Tecan Spark 10M plate reader. The amount of ammonia remaining in the media was quantified using EnzyChrom Ammonia/Ammonium Assay Kit (BioAssay Systems) and signal detected as above. Ammonia clearance was calculated by deducting the remaining amount of ammonium in the media from the amount of ammonium measured in a control well with no cells.

### MTT Assay

Huh7 cells stably expressing the mutant minigene were treated with SSOs at 10 nM, 50 nM or 100 nM by free uptake in calcium-enriched medium for 72 hours. MTT assay was then performed using the MTT Assay Kit (Abcam) as per manufacturer's instructions. In brief, cells were treated with MTT solution for 3 hours at 37° C., after which MTT solvent was added to the cells to release and dissolve the reduced formazan crystals. Signals were then measured using Tecan Spark 10M plate reader.

### Example 1

The sequences of SSOs are shown in Table 1 below. Every sugar moiety in a SSO is linked via a phosphorothioate backbone. Every sugar moiety in a SSO is modified with 2′-O-methyl or 2′-O-methoxyethyl, with the exception of nucleotides substituted with locked nucleic acid as indicated in bold font.

TABLE 1

| SEQ | Name of | |
|---|---|---|
| ID NO | SSO | Sequence from 5′ to 3′ |
| 1 | 1988 | UGACAGGCAUUCUCAUCAGUGAAAAGUAUUCUGU |
| 2 | 1989 | UAGGUGCUCAAUAAAUGACAGGCAUUCUCAUC |
| 3 | 1990 | UACAUGCAGCUCAGCAGAAUGCUUUA |
| 4 | 1991 | GCUCAGCAGAAUGCUUUAGAUGUAUAGUAGG |
| 5 | 2004 | UAUACAUGCAGCUCAGCAGAAUGCUUUAG |
| 6 | 2005 | AACCUUUUGAAGUCCUGUAAGCAUCUACUUACAU |
| | | UUA |
| 7 | 2006 | AUGAAUGAACAAAUCGUGAGCUAUUGAACAAUGG |
| | | GAGG |
| 8 | 2007 | GAUGUAUAGUAGGUGCUCAAUAAAUGACAGGCAU |
| | | U |
| 9 | 2008 | AGGUGCUCAAUAAAUGACAGGCAUUCUCAUCAG |
| 10 | 2032 | AAUAAAUGACAGGCAUUCUCAUCA |
| 11 | 2033 | AAUGACAGGCAUUCUCAUCAG |
| 12 | 2034 | AUGACAGGCAUUCUCAUCA |
| 13 | 2034.1 | AUGACAGGCAUUCUCAUCA |
| 14 | 2034.2 | AUGACAGGCAUUCUCAUCA |
| 15 | 2034.3 | AUGACAGGCAUUCUCAUCA |
| 16 | 2034.4 | AUGACAGGCAUUCUCAUCA |
| 17 | 2034.5 | AUGACAGGCAUUCUCAUCA |
| 18 | 2034.6 | ATGACAGGCAUUCTCAUCA |
| 19 | 2034.7 | ATGACAGGCAUUCTCATCA |
| 20 | 2034.8 | AUGACAGGCAUUCTCAUCA |
| 21 | 2034.9 | AUGACAGGCAUUCTCATCA |
| 22 | 2034.10 | ATGACAGGCAUUCUCAUCA |
| 23 | 2034.11 | ATGACAGGCAUUCUCAUCA |
| 24 | 2034.12 | ATGACAGGCAUUCUCATCA |
| 25 | 2034.13 | AUGACAGGCAUUCUCAUCA |
| 26 | 2034.14 | AUGACAGGCAUUCUCATCA |
| 27 | 2034.15 | ATGACAGGCAUUCUCATCA |

The SSO comprising the sequence of SEQ ID NO 12 has the same target sequence as the SSOs comprising the sequence of any one of SEQ ID NOs 13 to 27.

In some embodiments, the SSOs comprise multiple chemical modifications. The sequences of SSOs with chemical modifications are shown in Table 2 below.

Nucleotides with a 2′-O-Methyl RNA (2′OMe) are indicated with “m”. Nucleotides with a 2′-O-methoxyethyl (2′MOE) RNA are indicated with “/MOEr/”. 2′MOE-modified thymidine is used in place of 2′MOE-modified uridine. Nucleotides with a locked nucleic acid (LNA) are indicated with “+”. LNA-modified thymidine is used in place of LNA-modified uridine. Nucleotides joined by a phosphorothioate (PS) bond to the following nucleotide are indicated with “*”. It would be generally known to the person skilled in the art that the number of phosphorothioate bands is one less than the number of bases. “20M” indicates that the SSO is modified with 2′-O-methyl RNA. “2MOE” indicates that the SSO is modified with 2′-O-methoxyethyl RNA. “2OML” indicates that the SSO includes 2′-O-methyl RNA and locked nucleic acid modifications. “2MOL” indicates that the SSO includes 2′-O-methoxyethyl RNA and locked nucleic acid modifications.

TABLE 2

| SEQ | Name | |
|---|---|---|
| ID NO | of SSO | Sequence from 5′ to 3′ |
| 48 | 1988 | mU*mG*mA*mC*mA*mG*mG*mC*mA*mU*mU*mC*mU*mC*mA*mU*m |
| | 2OM | C*mA*mG*mU*mG*mA*mA*mA*mA*mG*mU*mA*mU*mU*mC*mU*mG* |
| | | mU |
| 49 | 1989 | mU*mA*mG*mG*mU*mG*mC*mU*mC*mA*mA*mU*mA*mA*mA*mU*m |
| | 2OM | G*mA*mC*mA*mG*mG*mC*mA*mU*mU*mC*mU*mC*mA*mU*mC |
| 50 | 1990 | mU*mA*mC*mA*mU*mG*mC*mA*mG*mC*mU*mC*mA*mG*mC*mA*m |
| | 2OM | G*mA*mA*mU*mG*mC*mU*mU*mU*mA |
| 51 | 1991 | mG*mC*mU*mC*mA*mG*mC*mA*mG*mA*mA*mU*mG*mC*mU*mU*mU*mA*m |
| | 2OM | G*mA*mU*mG*mU*mA*mU*mA*mG*mU*mA*mG*mG |
| 52 | 2004 | mU*mA*mU*mA*mC*mA*mU*mG*mC*mA*mG*mC*mU*mC*mA*mG*m |
| | 2OM | C*mA*mG*mA*mA*mU*mG*mC*mU*mU*mU*mA*mG |
| 53 | 2005 | mA*mA*mC*mC*mU*mU*mU*mU*mG*mA*mA*mG*mU*mC*mC*mU*m |
| | 2OM | G*mU*mA*mA*mG*mC*mA*mU*mC*mU*mA*mC*mU*mU*mA*mC*mA* |
| | | mU*mU*mU*mA |
| 54 | 2006 | mA*mU*mG*mA*mA*mU*mG*mA*mA*mC*mA*mA*mA*mU*mC*mG*mU |
| | 2OM | *mG*mA*mG*mC*mU*mA*mU*mU*mG*mA*mA*mC*mA*mA*mU*mG*m |
| | | G*mG*mA*mG*mG |
| 55 | 2007 | mG*mA*mU*mG*mU*mA*mU*mA*mG*mU*mA*mG*mG*mU*mG*mC*m |
| | 2OM | U*mC*mA*mA*mU*mA*mA*mA*mU*mG*mA*mC*mA*mG*mG*mC*mA* |
| | | mU*mU |
| 56 | 2008 | mA*mG*mG*mU*mG*mC*mU*mC*mA*mA*mU*mA*mA*mA*mU*mG*mA |
| | 2OM | *mC*mA*mG*mG*mC*mA*mU*mU*mC*mU*mC*mA*mU*mC*mA*mG |
| 57 | 2032 | mA*mA*mU*mA*mA*mA*mU*mG*mA*mC*mA*mG*mG*mC*mA*mU*mU |
| | 2OM | *mC*mU*mC*mA*mU*mC*mA |
| 58 | 2032 | /MOErA/*/MOErA/*/MOErT/*/MOErA/*/MOErA/*/MOErA/*/MOErT/*/MOErG/ |
| | 2MOE | */MOErA/*/MOErC/*/MOErA/*/MOErG/*/MOErG/*/MOErC/*/MOErA/*/ |
| | | MOErT/*/MOErT/*/MOErC/*/MOErT/*/MOErC/*/MOErA/*/MOErT/*/MOErC/ |
| | | */MOErA/ |
| 59 | 2033 | mA*mA*mU*mG*mA*mC*mA*mG*mG*mC*mA*mU*mU*mC*mU*mC*mA |
| | 2OM | *mU*mC*mA*mG |
| 60 | 2033 | /MOErA/*/MOErA/*/MOErT/*/MOErG/*/MOErA/*/MOErC/*/MOErA/*/MOErG/ |
| | 2MOE | */MOErG/*/MOErC/*/MOErA/*/MOErT/*/MOErT/*/MOErC/*/MOErT/*/ |
| | | MOErC/*/MOErA/*/MOErT/*/MOErC/*/MOErA/*/MOErG/ |
| 61 | 2034 | mA*mU*mG*mA*mC*mA*mG*mG*mC*mA*mU*mU*mC*mU*mC*mA*m |
| | 2OM | U*mC*mA |
| 62 | 2034 | /MOErA/*/MOErT/*/MOErG/*/MOErA/*/MOErC/*/MOErA/*/MOErG/*/MOErG/ |
| | 2MOE | */MOErC/*/MOErA/*/MOErT/*/MOErT/*/MOErC/*/MOErT/*/MOErC/*/ |
| | | MOErA/*/MOErT/*/MOErC/*/MOErA/ |
| 63 | 2034.1 | mA*mU* + G*mA*mC*mA* + G*mG*mC*mA*mU*mU*mC*mU*mC* + A*mU* |
| | 2OML | mC*mA |
| 64 | 2034.2 | mA*mU*mG*mA* + C*mA*mG*mG*mC*mA*mU*mU* + C*mU*mC* + A*mU* |
| | 2OML | mC*mA |
| 65 | 2034.3 | mA*mU*mG*mA* + C*mA*mG*mG*mC*mA*mU*mU* + C*mU* + C*mA*mU* |
| | 2OML | mC*mA |
| 66 | 2034.3 | /MOErA/*/MOErT/*/MOErG/*/MOErA/* + C*/MOErA/*/MOErG/*/MOErG/*/ |
| | 2MOL | MOErC/*/MOErA/*/MOErT/*/MOErT/* + C*/MOErT/* + C*/MOErA/*/MOErT/*/ |
| | | MOErC/*/MOErA/ |
| 67 | 2034.4 | + A*mU*mG*mA*mC*mA*mG*mG*mC*mA*mU*mU*mC*mU*mC* + A*mU* |
| | 2OML | mC* + A |
| 68 | 2034.4 | + A*/MOErT/*/MOErG/*/MOErA/*/MOErC/*/MOErA/*/MOErG/*/MOErG/*/ |
| | 2MOL | MOErC/*/MOErA/*/MOErT/*/MOErT/*/MOErC/*/MOErT/*/MOErC/* + A*/ |
| | | MOErT/*/MOErC/* + A |
| 69 | 2034.5 | + A*mU*mG* + A*mC*mA*mG*mG*mC*mA*mU*mU*mC*mU*mC* + A*mU* |
| | 2OML | mC* + A |
| 70 | 2034.5 | + A*/MOErT/*/MOErG/* + A*/MOErC/*/MOErA/*/MOErG/*/MOErG/*/MOErC/ |
| | 2MOL | */MOErA/*/MOErT/*/MOErT/*/MOErC/*/MOErT/*/MOErC/* + A*/MOErT/*/ |
| | | MOErC/* + A |
| 71 | 2034.6 | mA* + T*mG*mA*mC*mA*mG*mG*mC*mA*mU*mU*mC* + T*mC* + A*mU* |
| | 2OML | mC*mA |
| 72 | 2034.7 | mA* + T*mG*mA*mC*mA*mG*mG*mC*mA*mU*mU*mC* + T*mC*mA* + T* |
| | 2OML | mC*mA |
| 73 | 2034.7 | /MOErA/* + T*/MOErG/*/MOErA/*/MOErC/*/MOErA/*/MOErG/*/MOErG/*/MOErC/ |
| | 2MOL | */MOE?A/*/MOET/*/MOErT/*/MOErC/* + T*/MOErC/*/MOErA/* + T*/MOErC/*/ |
| | | MOErA/ |
| 74 | 2034.8 | mA*mU*mG*mA*mC*mA*mG*mG*mC*mA*mU*mU*mC* + T* + C* + A*mU* |
| | 2OML | mC*mA |
| 75 | 2034.9 | mA*mU*mG*mA*mC*mA*mG*mG*mC*mA*mU*mU*mC* + T* + C* + A* + T* |
| | 2OML | mC*mA |
| 76 | 2034.10 | + A* + T*mG*mA*mC*mA*mG*mG*mC*mA*mU*mU*mC*mU*mC*mA*mU* |
| | 2OML | mC* + A |
| 77 | 2034.10 | + A* + T*/MOErG/*/MOErA/*/MOErC/*/MOErA/*/MOErG/*/MOErG/*/MOErC/ |
| | 2MOL | */MOErA/*/MOErT/*/MOErT/*/MOErC/*/MOErT/*/MOErC/*/MOErA/*/ |
| | | MOErT/*/MOErC/* + A |
| 78 | 2034.11 | + A* + T*mG*mA*mC*mA*mG*mG*mC*mA*mU*mU*mC*mU*mC* + A*mU* |
| | 2OML | mC*mA |
| 79 | 2034.11 | + A* + T*/MOErG/*/MOErA/*/MOErC/*/MOErA/*/MOErG/*/MOErG/*/MOErC/ |
| | 2MOL | */MOErA/*/MOErT/*/MOErT/*/MOErC/*/MOErT/*/MOErC/* + A*/MOErT/*/ |
| | | MOErC/*/MOErA/ |
| 80 | 2034.12 | + A* + T*mG*mA*mC*mA*mG*mG*mC*mA*mU*mU*mC*mU*mC*mA* + T* |
| | 2OML | mC*mA |
| 81 | 2034.12 | + A* + T*/MOErG/*/MOErA/*/MOErC/*/MOErA/*/MOErG/*/MOErG/*/MOErC/ |
| | 2MOL | */MOErA/*/MOErT/*/MOErT/*/MOErC/*/MOErT/*/MOErC/*/MOErA/* + T*/ |
| | | MOErC/*/MOErA/ |
| 82 | 2034.13 | + A*mU*mG*mA*mC*mA*mG*mG*mC*mA*mU*mU*mC*mU*mC*mA*mU |
| | 2OML | * + C* + A |
| 83 | 2034.13 | + A*/MOErT/*/MOErG/*/MOErA/*/MOErC/*/MOErA/*/MOErG/*/MOErG/*/ |
| | 2MOL | MOErC/*/MOErA/*/MOErT/*/MOErT/*/MOErC/*/MOErT/*/MOErC/*/MOErA/ |
| | | */MOErT/* + C* + A |
| 84 | 2034.14 | + A*mU*mG*mA*mC*mA*mG*mG*mC*mA*mU*mU*mC*mU*mC*mA* + T* |
| | 2OML | mC* + A |
| 85 | 2034.14 | + A*/MOErT/*/MOErG/*/MOErA/*/MOErC/*/MOErA/*/MOErG/*/MOErG/*/ |
| | 2MOL | MOErC/*/MOErA/*/MOErT/*/MOErT/*/MOErC/*/MOErT/*/MOErC/*/MOErA/ |
| | | * + T*/MOErC/* + A |
| 86 | 2034.15 | mA* + T*mG*mA*mC*mA*mG*mG*mC*mA*mU*mU*mC*mU*mC*mA* + T* |
| | 2OML | mC* + A |
| 87 | 2034.15 | /MOErA/* + T*/MOErG/*/MOErA/*/MOErC/*/MOErA/*/MOErG/*/MOErG/*/ |
| | 2MOL | MOErC/*/MOErA/*/MOErT/*/MOErT/*/MOErC/*/MOErT/*/MOErC/*/MOErA/ |
| | | * + T*/MOErC/* + A |

### Example 2

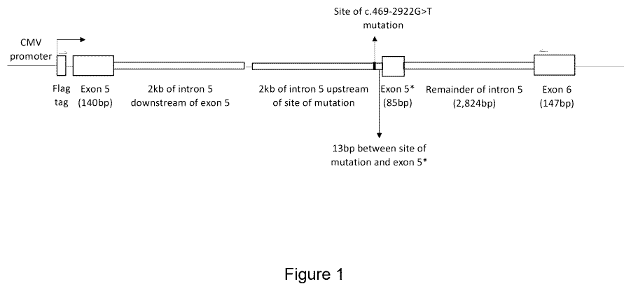

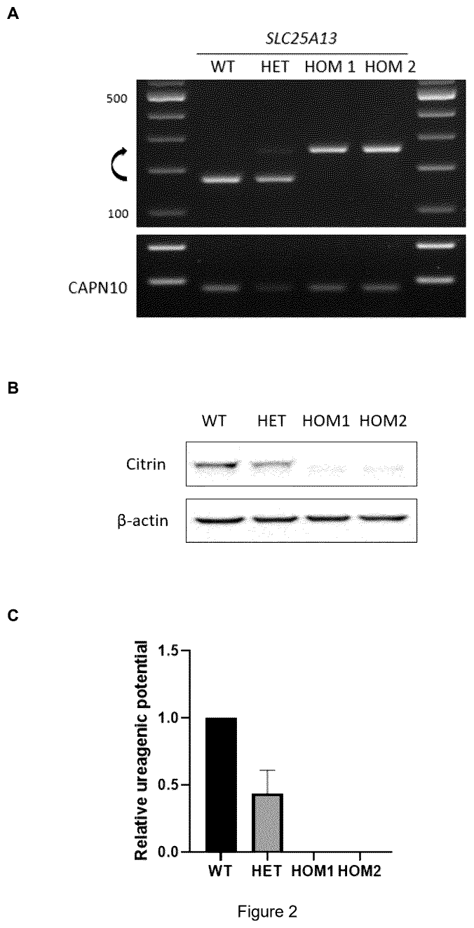

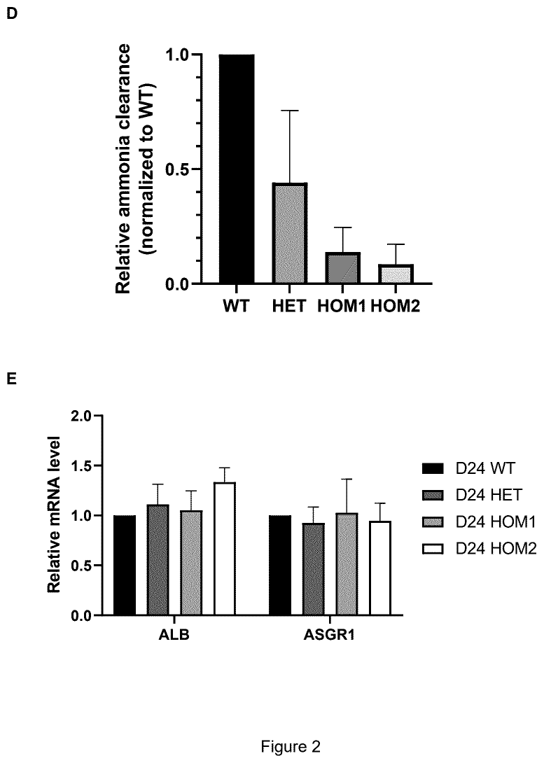

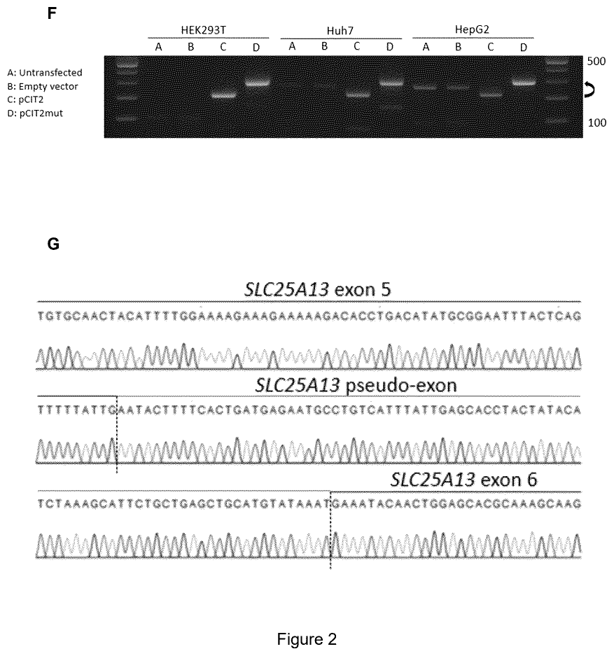

To facilitate the validation of the rationally designed SSOs, a minigene system that harbours the specific genomic mutation, c.469-2922G>T of the SLC25A13 gene, was constructed. As shown in FIG. 1, the minigene includes the full sequence of exon 5, the first 2,000 bases and last 4,923 bases of intron 5, and full sequence of exon 6; the relative loci of the G>T substitution is demarcated in the figure. In the wild type minigene, there was proper splicing of exon 5 to exon 6, whereas upon introduction of the c.469-2922G>T mutation, the inclusion of the pseudoexon (Exon 5* or SLC25A13-PE5) when the mutant minigene was expressed in several human cell lines was confirmed (FIG. 2). This would imply that the c.469-2922G>T mutation is causative for the retention of SLC25A13-PE5 in mature SLC25A13 transcripts. The loss of citrin protein expression and the subsequent loss of ureagenic potential and ammonia clearing capability in iHeps that carry the c.469-2922G>T mutation confirmed the pathogenic effects (FIGS. 2B, 2C and 2D).

### Example 3

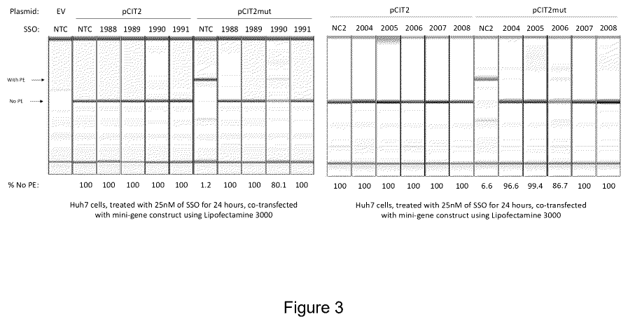

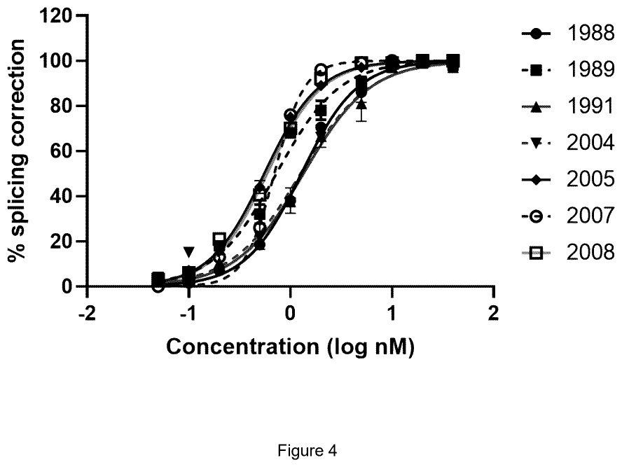

As the pseudoexon is 85 bases long, its inclusion shifts the codon reading frame of SLC25A13 transcripts, resulting in the loss of expression and protein function (FIG. 2). As a therapeutic strategy to restore the expression of the wildtype protein, 9 SSOs were designed to induce the exclusion of the pseudoexon and thereby corrects the reading frame. As shown in FIG. 3, each of the SSO were highly efficient in inducing SLC25A13-PE5 exon skipping. The 7 most efficient SSOs were selected and titration experiments were performed to obtain their respective concentration responses (FIG. 4), for the purpose of further differentiating the best performing SSO(s). Each of the SSOs were observed to achieve complete exclusion of the pseudoexon at a concentration lower than 10 nM that is, IC₁₀₀<10 nM. SSOs #2005, #2007 and #2008 were the top 3 performers whose IC₅₀<0.1 nM and IC₇₅˜0.1 nM.

### Example 4

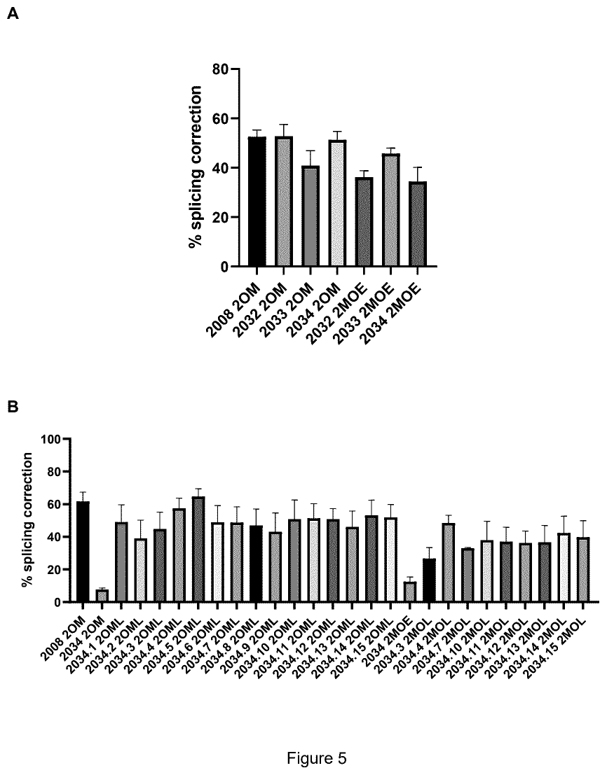

With the aim to reduce the molecular size of the lead SSO, which facilitates cellular uptake and lower cGMP manufacturing cost, shorter versions of SSO #2008 were rationally designed with substitution of locked nuclei acids (LNAs) at specific ribose. FIG. 5 shows that shortened SSOs, #2032, #2033 and #2034, which are 40% shorter than the parent #2008, lost substantially their efficiency in inducing the exclusion of the pseudoexon. #2034 was selected for further optimization with 15 permutations of mixed chemical modifications, labelled from #2034.1 to #2034.15. Every sugar moiety in a mixmer is either modified with 2′-O-methyl or substituted with a locked nucleic acid that is linked via phosphorothioate backbone. Restoration of the efficiency in inducing the exclusion of the pseudoexon was observed in several mixmers of #2034. In both sets of experiments, SSOs were co-transfected at 1 nM with 500 ng of pCIT2mut into Huh7. PCR was performed on complementary DNA generated from RNA extracted from the transfected cells. Capillary electrophoresis was used on PCR products to quantify the amount of products with the pseudoexon and without the pseudoexon. Efficacy is reflected as the percentage of PCR products without the pseudoexon over the total amount of PCR products (% splicing correction).

### Example 5

The efficiency in inducing the exclusion of SLC25A13-PE5 by each mixmer of #2034, labelled from #2034.1 to #2034.15, was determined by free uptake in calcium-enriched medium (CEM) on Huh7 or HepG2 cells stably expressing the mutant minigene when treated with 20 nM or 200 nM of SSOs, respectively. The use of CEM stimulates in vitro uptake by cells and better reflects the in vivo efficacy when compared to transfection. The parent #2008, which is 40% longer than #2034, abrograted almost all of its efficiency (FIG. 6A). By contrast, efficiency of the majority #2034 mixmers were retained, which suggests that SSO molecular size may be an important factor influencing kinetics of free uptake by cells (FIG. 6A). Subsequently, dose responses for two most efficient mixmers, #2034.5 and #2034.15, were obtained and compared with their parent #2008 and the singly 2′-O-methyl modified version (#2034). Both #2034.5 and #2034.15 mixmers manifest similar dose responses and are 10× more potent than either #2008 or #2034 (FIG. 6B); EC₅₀ for both #2034.5 and #2034.15 (<10′^{1.5} nM) are about 10 times lower than #2008 and #2034. FIG. 6C showed that no apparent toxicity effect was observed on cells treated with any of the SSOs.

### Example 6

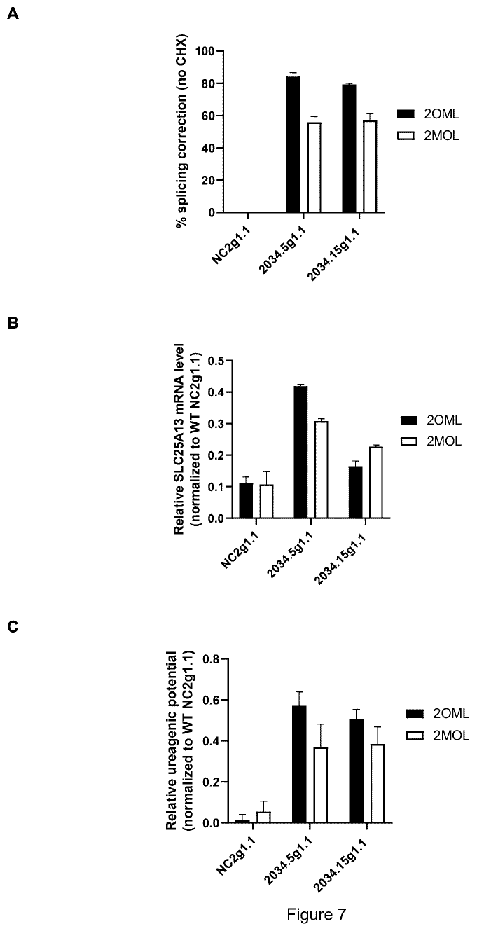

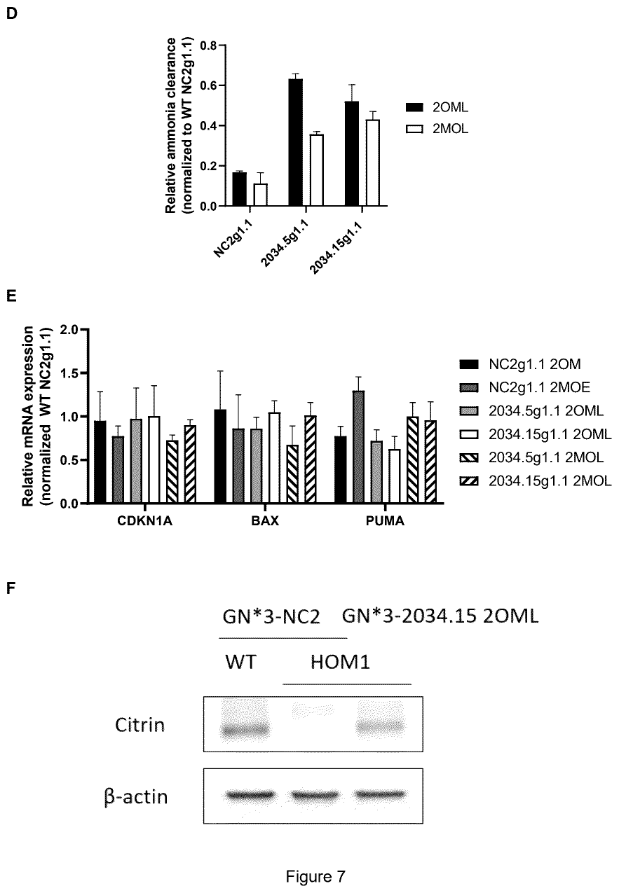

To demonstrate therapeutic application, lead mixmers were conjugated with three GaINAc molecules (GN*3) to simulate asialoglycoprotein receptor-mediated functional uptake (in the absence of CEM and a transfection agent) by iHeps homozygous for SLC25A13-PE5. The GN*3 moeity was chemically bonded in a trivalent configuration to the 5′ of each SSO, which allow for receptor-mediated uptake of the SSOs through the asialoglycoprotein receptor expressed specifically on hepatocytes. 4 μM of GaINAc*3 (GN*3)-conjugated non-targeting control (NC2 g1.1), 2034.5 (2034.5g1.1) or 2034.15 (2034.15g1.1) in either 2′-O-methyl+LNA (2OML) or 2′-O-methoxyethyl+LNA (2MOL) chemistry combinations were incubated on cultured iHeps. FIG. 7A reveals that the GaINAc-conjugated SSOs 2034.5 and 2034.15, but not N2, can modulate splice-out of SLC25A13-PE5 from endogenous SLC25A13 transcripts, which subsequently rescues SLC25A13 transcript levels that would otherwise be degraded by nonsense-mediated decay in mutant iHeps (FIG. 71B). The restoration of the full-length wildtype SLC25A13 expression in mutant iHeps, by either 2034.5g1.1 or 2034.15g1.1, rescues ureagenesis (FIG. 70) that leads to restoration of ammonia clearance (FIG. 7D). The rescue in functional protein activity corroborated with the rescue in the full length citrin protein expression level via immunoblot in mutant iHeps treated with 2034.15g1.1 modified in 2OML chemistry combination (FIG. 7F). No acute toxicity was observed in treated iHeps from 2034.5g1.1 and 2034.15g1.1 in 2OML or 2MOL chemistry combinations, as inferred from no significant increase in expression of acute toxicity markers CDKN1A, BAX and PUMA (FIG. 7E). The sequences of primers used in quantitative real-time PCR for the quantitation of acute toxicity markers CDKN1A, BAX and PUMA, hepatocyte difAerentiation markers ALB and ASGR1, SLC25A13 and the loading control CAPN1 mRNA, are shown in Table 3.

TABLE 3

| SEQ | | |
|---|---|---|
| ID NO | Description | Sequence from 5′ to 3′ |
| 30 | CDKN1A forward | AGCAGAGGAAGACCATGTGGA |
| | primer | |
| 31 | CDKN1A reverse | AATGTGTCATGOTGGTCTGCC |
| | primer | |
| 32 | BAX forward primer | CCCGAGAGGTCTTTTTCCGAG |
| 33 | BAX reverse primer | CCAGCCCATGATGGTTCTGAT |
| 34 | PUMA forward | GACCTCAACGCACAGTACGAG |
| | primer | |
| 35 | PUMA reverse | AGGAGTCCCATGATGAGATTGT |
| | primer | |
| 36 | ALB forward primer | GTTGCATGAGAAAACGCCAGT |
| 37 | ALB reverse primer | GTCGCCTGTTCACCAAGGAT |
| 38 | ASGR1 forward | GAGACAGAGCTGGACAAG |
| | primer | |
| 39 | ASGR1 reverse | CCCCTTCCOTTAAAATCCT |
| | primer | |
| 40 | SLC25A13 forward | TGGACTGTATAGAGGTCTGTTGC |
| | primer | |
| 41 | SLC25A13 reverse | CCCTCACAAAATCGTTCACTGT |
| | primer | |
| 42 | CAPN10 forward | CTTCTGCGACTTGTOTACGCC |
| | primer | |
| 43 | CAPN10 reverse | GTGTGGCACAAATCTCCTGG |
| | primer | |

Whilst there has been described in the foregoing description preferred embodiments of the present invention, it will be understood by those skilled in the technology concerned that many variations or modifications in details of design or construction may be made without departing from the present invention.

1. A method of exon-skipping comprising providing a splice-switching oligonucleotide (SSO) that binds to a site within a target region present on a pre-mRNA transcript of the SLC25A13 gene, wherein the binding of the SSO induces the exclusion of SLC25A13-PE5 from a mature mRNA transcript of the SLC25A13 gene.

2. The method of claim 1, wherein the target region having at least 95% sequence identity to SEQ ID NO: 28.

3. The method of claims 1 or 2, wherein SLC25A13-PE5 comprises the sequence of SEQ ID NO: 29.

4. The method of any one of claims 1 to 3, comprising providing an SSO having a binding site that lies within SLC25A13-PE5.

5. The method of any one of claims 1 to 3, comprising providing an SSO having a binding site that overlaps with the acceptor splice site of SLC25A13-PE5 and with or a part of SLC25A13-PE5.

6. The method of any one of claims 1 to 3, comprising providing an SSO having a binding site that overlaps with or a part of SLC25A13-PE5 and with the donor splice site of SLC25A13-PE5.

7. The method of any one of claims 1 to 3, comprising providing an SSO having a sequence selected from the group consisting of SEQ ID NOs 1 to 12.

8. The method of any one of claims 1 to 3, comprising providing an SSO having a sequence selected from the group consisting of SEQ ID NOs 13 to 27.

9. A splice-switching oligonucleotide (SSO) that binds to a site within a target region present on a pre-mRNA transcript of the SLC25A13 gene, the target region having at least 95% sequence identity to SEQ ID NO: 28, and wherein binding of the SSO induces the exclusion of SLC25A13-PE5 from a mature mRNA transcript of the SLC25A13 gene.

10. The SSO of claim 9, wherein the SSO has a binding site that lies within SLC25A13-PE5 and wherein SLC25A13-PE5 comprises the sequence of SEQ ID NO: 29.

11. The SSO of claim 9, wherein the SSO has a binding site that overlaps with the acceptor splice site of SLC25A13-PE5 and with or a part of SLC25A13-PE5, and wherein SLC25A13-PE5 comprises the sequence of SEQ ID NO: 29.

12. The SSO of claim 9, wherein the SSO has a binding site that overlaps with or a part of SLC25A13-PE5 and with the donor splice site of SLC25A13-PE5, and wherein SLC25A13-PE5 comprises the sequence of SEQ ID NO: 29.

13. The SSO of claim 9, comprising a sequence selected from the group consisting of SEQ ID NOs 1 to 12.

14. The SSO of claim 9, comprising a sequence selected from the group consisting of SEQ ID NOs 13 to 27.

15. An SSO of any one of claims 9 to 14 for use in treating citrin deficiency.

16. Use of an SSO of any one of claims 9 to 14 in the manufacture of a medicament for treating citrin deficiency.

17. A method of treating citrin deficiency comprising administering to a subject a composition comprising an SSO according to any one of claims 9 to 14.

18. The method of any one of claims 1 to 8, or the SSO of any one of claims 9 to 15, or the use of claim 16 or the method of claim 17, wherein the SSO is between 15 and 40 nucleotides in length.

19. The method of any one of claims 1 to 8 and 18, or the SSO of any one of claims 9 to 15 and 18, or the use of claims 16 or 18 or the method of claims 17 or 18, wherein at least one of the nucleotides of the SSO is chemically modified and wherein the chemical modification is 2′-O-methyl RNA modification, 2′-O-methoxyethyl RNA modification, locked nucleic acid substitution, or phosphorothioate linkage.

20. The method of any one of claims 1 to 8 and 18 to 19, or the SSO of any one of claims 9 to 15 and 18 to 19, or the use of any one of claims 16 or 18 to 19, or the method of any one of claims 17 to 19, wherein the SSO comprises phosphorothioate linkages between all nucleotides of the SSO.

21. The method of any one of claims 1 to 8 and 18 to 20, or the SSO of any one of claims 9 to 15 and 18 to 20, or the use of any one of claims 16 or 18 to 20, or the method of any one of claims 17 to 20, wherein each nucleotide of the SSO comprises either a 2′-O-methyl RNA modification, a 2′-O-methoxyethyl RNA modification or a locked nucleic acid substitution.

22. A pharmaceutical composition comprising (a) a therapeutically effective amount of an SSO according to any one of claims 9 to 14 and 18 to 21 and (b) one or more pharmaceutically acceptable carriers and/or diluents.


SEQUENCE LISTING (87 sequences)

SEQ ID NO: 1 - RNA, 34 nt, synthetic construct
  source 1..34: mol_type = other RNA
tgacaggcattctcatcagtgaaaagtattctgt

SEQ ID NO: 2 - RNA, 32 nt, synthetic construct
  source 1..32: mol_type = other RNA
taggtgctcaataaatgacaggcattctcatc

SEQ ID NO: 3 - RNA, 26 nt, synthetic construct
  source 1..26: mol_type = other RNA
tacatgcagctcagcagaatgcttta

SEQ ID NO: 4 - RNA, 31 nt, synthetic construct
  source 1..31: mol_type = other RNA
gctcagcagaatgctttagatgtatagtagg

SEQ ID NO: 5 - RNA, 29 nt, synthetic construct
  source 1..29: mol_type = other RNA
tatacatgcagctcagcagaatgctttag

SEQ ID NO: 6 - RNA, 37 nt, synthetic construct
  source 1..37: mol_type = other RNA
aaccttttgaagtcctgtaagcatctacttacattta

SEQ ID NO: 7 - RNA, 38 nt, synthetic construct
  source 1..38: mol_type = other RNA
atgaatgaacaaatcgtgagctattgaacaatgggagg

SEQ ID NO: 8 - RNA, 35 nt, synthetic construct
  source 1..35: mol_type = other RNA
gatgtatagtaggtgctcaataaatgacaggcatt

SEQ ID NO: 9 - RNA, 33 nt, synthetic construct
  source 1..33: mol_type = other RNA
aggtgctcaataaatgacaggcattctcatcag

SEQ ID NO: 10 - RNA, 24 nt, synthetic construct
  source 1..24: mol_type = other RNA
aataaatgacaggcattctcatca

SEQ ID NO: 11 - RNA, 21 nt, synthetic construct
  source 1..21: mol_type = other RNA
aatgacaggcattctcatcag

SEQ ID NO: 12 - RNA, 19 nt, synthetic construct
  source 1..19: mol_type = other RNA
atgacaggcattctcatca

SEQ ID NO: 13 - RNA, 19 nt, synthetic construct
  source 1..19: mol_type = other RNA
  modified_base 3: mod_base = OTHER; note = locked nucleic acid
  modified_base 7: mod_base = OTHER; note = locked nucleic acid
  modified_base 16: mod_base = OTHER; note = locked nucleic acid
atgacaggcattctcatca

SEQ ID NO: 14 - RNA, 19 nt, synthetic construct
  source 1..19: mol_type = other RNA
  modified_base 5: mod_base = OTHER; note = locked nucleic acid
  modified_base 13: mod_base = OTHER; note = locked nucleic acid
  modified_base 16: mod_base = OTHER; note = locked nucleic acid
atgacaggcattctcatca

SEQ ID NO: 15 - RNA, 19 nt, synthetic construct
  source 1..19: mol_type = other RNA
  modified_base 5: mod_base = OTHER; note = locked nucleic acid
  modified_base 13: mod_base = OTHER; note = locked nucleic acid
  modified_base 15: mod_base = OTHER; note = locked nucleic acid
atgacaggcattctcatca

SEQ ID NO: 16 - RNA, 19 nt, synthetic construct
  source 1..19: mol_type = other RNA
  modified_base 1: mod_base = OTHER; note = locked nucleic acid
  modified_base 16: mod_base = OTHER; note = locked nucleic acid
  modified_base 19: mod_base = OTHER; note = locked nucleic acid
atgacaggcattctcatca

SEQ ID NO: 17 - RNA, 19 nt, synthetic construct
  source 1..19: mol_type = other RNA
  modified_base 1: mod_base = OTHER; note = locked nucleic acid
  modified_base 4: mod_base = OTHER; note = locked nucleic acid
  modified_base 16: mod_base = OTHER; note = locked nucleic acid
  modified_base 19: mod_base = OTHER; note = locked nucleic acid
atgacaggcattctcatca

SEQ ID NO: 18 - RNA, 19 nt, synthetic construct
  source 1..19: mol_type = other RNA
  modified_base 2: mod_base = OTHER; note = locked nucleic acid
  modified_base 14: mod_base = OTHER; note = locked nucleic acid
  modified_base 16: mod_base = OTHER; note = locked nucleic acid
  modified_base 2: mod_base = OTHER; note = thymine
  modified_base 14: mod_base = OTHER; note = thymine
atgacaggcattctcatca

SEQ ID NO: 19 - RNA, 19 nt, synthetic construct
  source 1..19: mol_type = other RNA
  modified_base 2: mod_base = OTHER; note = locked nucleic acid
  modified_base 14: mod_base = OTHER; note = locked nucleic acid
  modified_base 17: mod_base = OTHER; note = locked nucleic acid
  modified_base 2: mod_base = OTHER; note = thymine
  modified_base 14: mod_base = OTHER; note = thymine
  modified_base 17: mod_base = OTHER; note = thymine
atgacaggcattctcatca

SEQ ID NO: 20 - RNA, 19 nt, synthetic construct
  source 1..19: mol_type = other RNA
  modified_base 14..16: mod_base = OTHER; note = locked nucleic acid
  modified_base 14: mod_base = OTHER; note = thymine
atgacaggcattctcatca

SEQ ID NO: 21 - RNA, 19 nt, synthetic construct
  source 1..19: mol_type = other RNA
  modified_base 14..17: mod_base = OTHER; note = locked nucleic acid
  modified_base 14: mod_base = OTHER; note = thymine
  modified_base 17: mod_base = OTHER; note = thymine
atgacaggcattctcatca

SEQ ID NO: 22 - RNA, 19 nt, synthetic construct
  source 1..19: mol_type = other RNA
  modified_base 1..2: mod_base = OTHER; note = locked nucleic acid
  modified_base 19: mod_base = OTHER; note = locked nucleic acid
  modified_base 2: mod_base = OTHER; note = thymine
atgacaggcattctcatca

SEQ ID NO: 23 - RNA, 19 nt, synthetic construct
  source 1..19: mol_type = other RNA
  modified_base 1..2: mod_base = OTHER; note = locked nucleic acid
  modified_base 16: mod_base = OTHER; note = locked nucleic acid
  modified_base 2: mod_base = OTHER; note = thymine
atgacaggcattctcatca

SEQ ID NO: 24 - RNA, 19 nt, synthetic construct
  source 1..19: mol_type = other RNA
  modified_base 1..2: mod_base = OTHER; note = locked nucleic acid
  modified_base 17: mod_base = OTHER; note = locked nucleic acid
  modified_base 2: mod_base = OTHER; note = thymine
  modified_base 17: mod_base = OTHER; note = thymine
atgacaggcattctcatca

SEQ ID NO: 25 - RNA, 19 nt, synthetic construct
  source 1..19: mol_type = other RNA
  modified_base 1: mod_base = OTHER; note = locked nucleic acid
  modified_base 18..19: mod_base = OTHER; note = locked nucleic acid
atgacaggcattctcatca

SEQ ID NO: 26 - RNA, 19 nt, synthetic construct
  source 1..19: mol_type = other RNA
  modified_base 1: mod_base = OTHER; note = locked nucleic acid
  modified_base 17: mod_base = OTHER; note = locked nucleic acid
  modified_base 19: mod_base = OTHER; note = locked nucleic acid
  modified_base 17: mod_base = OTHER; note = thymine
atgacaggcattctcatca

SEQ ID NO: 27 - RNA, 19 nt, synthetic construct
  source 1..19: mol_type = other RNA
  modified_base 2: mod_base = OTHER; note = locked nucleic acid
  modified_base 17: mod_base = OTHER; note = locked nucleic acid
  modified_base 19: mod_base = OTHER; note = locked nucleic acid
  modified_base 2: mod_base = OTHER; note = thymine
  modified_base 17: mod_base = OTHER; note = thymine
atgacaggcattctcatca

SEQ ID NO: 28 - RNA, 185 nt, Homo sapiens
  source 1..185: mol_type = mRNA
cctcccattgttcaatagctcacgatttgttcattcatttggttttacagaatacttttcactgatgagaatgcctgtcatttattgagcacctactatacatctaaagcattctgctgagctgcatgtataaatgtaagtagatgcttacaggacttcaaaaggttatactgtcttttccttgg

SEQ ID NO: 29 - RNA, 85 nt, Homo sapiens
  source 1..85: mol_type = mRNA
aatacttttcactgatgagaatgcctgtcatttattgagcacctactatacatctaaagcattctgctgagctgcatgtataaat

SEQ ID NO: 30 - DNA, 21 bp, synthetic construct
  source 1..21: mol_type = other DNA
agcagaggaagaccatgtgga

SEQ ID NO: 31 - DNA, 21 bp, synthetic construct
  source 1..21: mol_type = other DNA
aatctgtcatgctggtctgcc

SEQ ID NO: 32 - DNA, 21 bp, synthetic construct
  source 1..21: mol_type = other DNA
cccgagaggtctttttccgag

SEQ ID NO: 33 - DNA, 21 bp, synthetic construct
  source 1..21: mol_type = other DNA
ccagcccatgatggttctgat

SEQ ID NO: 34 - DNA, 21 bp, synthetic construct
  source 1..21: mol_type = other DNA
gacctcaacgcacagtacgag

SEQ ID NO: 35 - DNA, 22 bp, synthetic construct
  source 1..22: mol_type = other DNA
aggagtcccatgatgagattgt

SEQ ID NO: 36 - DNA, 21 bp, synthetic construct
  source 1..21: mol_type = other DNA
gttgcatgagaaaacgccagt

SEQ ID NO: 37 - DNA, 20 bp, synthetic construct
  source 1..20: mol_type = other DNA
gtcgcctgttcaccaaggat

SEQ ID NO: 38 - DNA, 18 bp, synthetic construct
  source 1..18: mol_type = other DNA
gagacagagctggacaag

SEQ ID NO: 39 - DNA, 19 bp, synthetic construct
  source 1..19: mol_type = other DNA
ccccttcccttaaaatcct

SEQ ID NO: 40 - DNA, 23 bp, synthetic construct
  source 1..23: mol_type = other DNA
tggactgtatagaggtctgttgc

SEQ ID NO: 41 - DNA, 22 bp, synthetic construct
  source 1..22: mol_type = other DNA
ccctcacaaaatcgttcactgt

SEQ ID NO: 42 - DNA, 21 bp, synthetic construct
  source 1..21: mol_type = other DNA
cttctgcgacttgtctacgcc

SEQ ID NO: 43 - DNA, 20 bp, synthetic construct
  source 1..20: mol_type = other DNA
gtgtggcacaaatctcctgg

SEQ ID NO: 44 - DNA, 185 bp, Homo sapiens
  source 1..185: mol_type = other DNA
cctcccattgttcaatagctcacgatttgttcattcatttggttttacagaatacttttcactgatgagaatgcctgtcatttattgagcacctactatacatctaaagcattctgctgagctgcatgtataaatgtaagtagatgcttacaggacttcaaaaggttatactgtcttttccttgg

SEQ ID NO: 45 - DNA, 85 bp, Homo sapiens
  source 1..85: mol_type = other DNA
aatacttttcactgatgagaatgcctgtcatttattgagcacctactatacatctaaagcattctgctgagctgcatgtataaat

SEQ ID NO: 46 - DNA, 16 bp, synthetic construct
  source 1..16: mol_type = other DNA
  modified_base 1: mod_base = OTHER; note = 5-methylcytosine
  modified_base 14: mod_base = OTHER; note = 5-methylcytosine
  modified_base 1..3: mod_base = OTHER; note = locked nucleic acid
  modified_base 14..16: mod_base = OTHER; note = locked nucleic acid
  modified_base 1..16: mod_base = OTHER; note = phosphorothioate bond
caaaggaaacacacat

SEQ ID NO: 47 - DNA, 16 bp, synthetic construct
  source 1..16: mol_type = other DNA
  modified_base 1: mod_base = OTHER; note = 5-methylcytosine
  modified_base 3: mod_base = OTHER; note = 5-methylcytosine
  modified_base 14: mod_base = OTHER; note = 5-methylcytosine
  modified_base 1..3: mod_base = OTHER; note = locked nucleic acid
  modified_base 14..16: mod_base = OTHER; note = locked nucleic acid
  modified_base 1..16: mod_base = OTHER; note = phosphorothioate bond
cacattccttgctctg

SEQ ID NO: 48 - RNA, 34 nt, synthetic construct; identical to SEQ ID NO: 1
  source 1..34: mol_type = other RNA
  modified_base 1..34: mod_base = OTHER; note = phosphorothioate bond; note = 2'-O-methyl

SEQ ID NO: 49 - RNA, 32 nt, synthetic construct; identical to SEQ ID NO: 2
  source 1..32: mol_type = other RNA
  modified_base 1..32: mod_base = OTHER; note = phosphorothioate bond; note = 2'-O-methyl

SEQ ID NO: 50 - RNA, 26 nt, synthetic construct
  source 1..26: mol_type = other RNA
  modified_base 1..26: mod_base = OTHER; note = 2'-O-methyl; note = phosphorothioate bond
tacatgcagctcagcagaatgcttta

SEQ ID NO: 51 - RNA, 31 nt, synthetic construct; identical to SEQ ID NO: 4
  source 1..31: mol_type = other RNA
  modified_base 1..31: mod_base = OTHER; note = phosphorothioate bond; note = 2'-O-methyl

SEQ ID NO: 52 - RNA, 29 nt, synthetic construct
  source 1..29: mol_type = other RNA
  modified_base 1..29: mod_base = OTHER; note = phosphorothioate bond; note = 2'-O-methyl
tatacatgcagctcagcagaatgctttag

SEQ ID NO: 53 - RNA, 37 nt, synthetic construct; identical to SEQ ID NO: 6
  source 1..37: mol_type = other RNA
  modified_base 1..37: mod_base = OTHER; note = phosphorothioate bond; note = 2'-O-methyl

SEQ ID NO: 54 - RNA, 38 nt, synthetic construct; identical to SEQ ID NO: 7
  source 1..38: mol_type = other RNA
  modified_base 1..38: mod_base = OTHER; note = 2'-O-methyl; note = phosphorothioate bond

SEQ ID NO: 55 - RNA, 35 nt, synthetic construct; identical to SEQ ID NO: 8
  source 1..35: mol_type = other RNA
  modified_base 1..35: mod_base = OTHER; note = phosphorothioate bond; note = 2'-O-methyl

SEQ ID NO: 56 - RNA, 33 nt, synthetic construct; identical to SEQ ID NO: 9
  source 1..33: mol_type = other RNA
  modified_base 1..33: mod_base = OTHER; note = 2'-O-methyl; note = phosphorothioate bond

SEQ ID NO: 57 - RNA, 24 nt, synthetic construct
  source 1..24: mol_type = other RNA
  modified_base 1..24: mod_base = OTHER; note = phosphorothioate bond; note = 2'-O-methyl
aataaatgacaggcattctcatca

SEQ ID NO: 58 - RNA, 24 nt, synthetic construct
  source 1..24: mol_type = other RNA
  modified_base 1..24: mod_base = OTHER; note = phosphorothioate bond; note = 2-O-methoxyethyl
aataaatgacaggcattctcatca

SEQ ID NO: 59 - RNA, 21 nt, synthetic construct
  source 1..21: mol_type = other RNA
  modified_base 1..21: mod_base = OTHER; note = phosphorothioate bond; note = 2'-O-methyl
aatgacaggcattctcatcag

SEQ ID NO: 60 - RNA, 21 nt, synthetic construct
  source 1..21: mol_type = other RNA
  modified_base 1..21: mod_base = OTHER; note = phosphorothioate bond; note = 2-O-methoxyethyl
aatgacaggcattctcatcag

SEQ ID NO: 61 - RNA, 19 nt, synthetic construct
  source 1..19: mol_type = other RNA
  modified_base 1..19: mod_base = OTHER; note = phosphorothioate bond; note = 2'-O-methyl
atgacaggcattctcatca

SEQ ID NO: 62 - RNA, 19 nt, synthetic construct
  source 1..19: mol_type = other RNA
  modified_base 1..19: mod_base = OTHER; note = phosphorothioate bond; note = 2-O-methoxyethyl
atgacaggcattctcatca

SEQ ID NO: 63 - RNA, 19 nt, synthetic construct
  source 1..19: mol_type = other RNA
  modified_base 1..19: mod_base = OTHER; note = phosphorothioate bond
  modified_base 3: mod_base = OTHER; note = locked nucleic acid
  modified_base 7: mod_base = OTHER; note = locked nucleic acid
  modified_base 16: mod_base = OTHER; note = locked nucleic acid
  modified_base 17..19: mod_base = OTHER; note = 2'-O-methyl
  modified_base 1..2: mod_base = OTHER; note = 2'-O-methyl
  modified_base 4..6: mod_base = OTHER; note = 2'-O-methyl
atgacaggcattctcatca

SEQ ID NO: 64 - RNA, 19 nt, synthetic construct
  source 1..19: mol_type = other RNA
  modified_base 1..19: mod_base = OTHER; note = phosphorothioate bond
  modified_base 16: mod_base = OTHER; note = locked nucleic acid
  modified_base 5: mod_base = OTHER; note = locked nucleic acid
  modified_base 13: mod_base = OTHER; note = locked nucleic acid
  modified_base 1..4: mod_base = OTHER; note = 2'-O-methyl
  modified_base 6..12: mod_base = OTHER; note = 2'-O-methyl
  modified_base 17..19: mod_base = OTHER; note = 2'-O-methyl
  modified_base 14..15: mod_base = OTHER; note = 2'-O-methyl
atgacaggcattctcatca

SEQ ID NO: 65 - RNA, 19 nt, synthetic construct
  source 1..19: mol_type = other RNA
  modified_base 1..19: mod_base = OTHER; note = phosphorothioate bond
  modified_base 5: mod_base = OTHER; note = locked nucleic acid
  modified_base 13: mod_base = OTHER; note = locked nucleic acid
  modified_base 15: mod_base = OTHER; note = locked nucleic acid
  modified_base 1..4: mod_base = OTHER; note = 2'-O-methyl
  modified_base 6..12: mod_base = OTHER; note = 2'-O-methyl
  modified_base 16..19: mod_base = OTHER; note = 2'-O-methyl
  modified_base 14: mod_base = OTHER; note = 2'-O-methyl
atgacaggcattctcatca

SEQ ID NO: 66 - RNA, 19 nt, synthetic construct
  source 1..19: mol_type = other RNA
  modified_base 1..19: mod_base = OTHER; note = phosphorothioate bond
  modified_base 5: mod_base = OTHER; note = locked nucleic acid
  modified_base 13: mod_base = OTHER; note = locked nucleic acid
  modified_base 15: mod_base = OTHER; note = locked nucleic acid
  modified_base 1..4: mod_base = OTHER; note = 2-O-methoxyethyl
  modified_base 6..12: mod_base = OTHER; note = 2-O-methoxyethyl
  modified_base 16..19: mod_base = OTHER; note = 2-O-methoxyethyl
  modified_base 14: mod_base = OTHER; note = 2-O-methoxyethyl
atgacaggcattctcatca

SEQ ID NO: 67 - RNA, 19 nt, synthetic construct
  source 1..19: mol_type = other RNA
  modified_base 1..19: mod_base = OTHER; note = phosphorothioate bond
  modified_base 16: mod_base = OTHER; note = locked nucleic acid
  modified_base 1: mod_base = OTHER; note = locked nucleic acid
  modified_base 19: mod_base = OTHER; note = locked nucleic acid
  modified_base 2..15: mod_base = OTHER; note = 2'-O-methyl
  modified_base 17..18: mod_base = OTHER; note = 2'-O-methyl
atgacaggcattctcatca

SEQ ID NO: 68 - RNA, 19 nt, synthetic construct
  source 1..19: mol_type = other RNA
  modified_base 1..19: mod_base = OTHER; note = phosphorothioate bond
  modified_base 16: mod_base = OTHER; note = locked nucleic acid
  modified_base 1: mod_base = OTHER; note = locked nucleic acid
  modified_base 19: mod_base = OTHER; note = locked nucleic acid
  modified_base 2..15: mod_base = OTHER; note = 2-O-methoxyethyl
  modified_base 17..18: mod_base = OTHER; note = 2-O-methoxyethyl
atgacaggcattctcatca

SEQ ID NO: 69 - RNA, 19 nt, synthetic construct
  source 1..19: mol_type = other RNA
  modified_base 1..19: mod_base = OTHER; note = phosphorothioate bond
  modified_base 16: mod_base = OTHER; note = locked nucleic acid
  modified_base 1: mod_base = OTHER; note = locked nucleic acid
  modified_base 4: mod_base = OTHER; note = locked nucleic acid
  modified_base 19: mod_base = OTHER; note = locked nucleic acid
  modified_base 2..3: mod_base = OTHER; note = 2'-O-methyl
  modified_base 5..15: mod_base = OTHER; note = 2'-O-methyl
  modified_base 17..18: mod_base = OTHER; note = 2'-O-methyl
atgacaggcattctcatca

SEQ ID NO: 70 - RNA, 19 nt, synthetic construct
  source 1..19: mol_type = other RNA
  modified_base 1..19: mod_base = OTHER; note = phosphorothioate bond
  modified_base 16: mod_base = OTHER; note = locked nucleic acid
  modified_base 1: mod_base = OTHER; note = locked nucleic acid
  modified_base 4: mod_base = OTHER; note = locked nucleic acid
  modified_base 19: mod_base = OTHER; note = locked nucleic acid
  modified_base 2..3: mod_base = OTHER; note = 2-O-methoxyethyl
  modified_base 5..15: mod_base = OTHER; note = 2-O-methoxyethyl
  modified_base 17..18: mod_base = OTHER; note = 2-O-methoxyethyl
atgacaggcattctcatca

SEQ ID NO: 71 - RNA, 19 nt, synthetic construct
  source 1..19: mol_type = other RNA
  modified_base 1..19: mod_base = OTHER; note = phosphorothioate bond
  modified_base 16: mod_base = OTHER; note = locked nucleic acid
  modified_base 2: mod_base = OTHER; note = locked nucleic acid
  modified_base 14: mod_base = OTHER; note = locked nucleic acid
  modified_base 17..19: mod_base = OTHER; note = 2'-O-methyl
  modified_base 3..13: mod_base = OTHER; note = 2'-O-methyl
  modified_base 1: mod_base = OTHER; note = 2'-O-methyl
  modified_base 15: mod_base = OTHER; note = 2'-O-methyl
atgacaggcattctcatca

SEQ ID NO: 72 - RNA, 19 nt, synthetic construct
  source 1..19: mol_type = other RNA
  modified_base 1..19: mod_base = OTHER; note = phosphorothioate bond
  modified_base 2: mod_base = OTHER; note = locked nucleic acid
  modified_base 14: mod_base = OTHER; note = locked nucleic acid
  modified_base 17: mod_base = OTHER; note = locked nucleic acid
  modified_base 3..13: mod_base = OTHER; note = 2'-O-methyl
  modified_base 1: mod_base = OTHER; note = 2'-O-methyl
  modified_base 18..19: mod_base = OTHER; note = 2'-O-methyl
  modified_base 15..16: mod_base = OTHER; note = 2'-O-methyl
atgacaggcattctcatca

SEQ ID NO: 73 - RNA, 19 nt, synthetic construct
  source 1..19: mol_type = other RNA
  modified_base 1..19: mod_base = OTHER; note = phosphorothioate bond
  modified_base 2: mod_base = OTHER; note = locked nucleic acid
  modified_base 14: mod_base = OTHER; note = locked nucleic acid
  modified_base 17: mod_base = OTHER; note = locked nucleic acid
  modified_base 18..19: mod_base = OTHER; note = 2-O-methoxyethyl
  modified_base 3..13: mod_base = OTHER; note = 2-O-methoxyethyl
  modified_base 15..16: mod_base = OTHER; note = 2-O-methoxyethyl
  modified_base 1: mod_base = OTHER; note = 2-O-methoxyethyl
atgacaggcattctcatca

SEQ ID NO: 74 - RNA, 19 nt, synthetic construct
  source 1..19: mol_type = other RNA
  modified_base 1..19: mod_base = OTHER; note = phosphorothioate bond
  modified_base 14..16: mod_base = OTHER; note = locked nucleic acid
  modified_base 17..19: mod_base = OTHER; note = 2'-O-methyl
  modified_base 1..13: mod_base = OTHER; note = 2'-O-methyl
atgacaggcattctcatca

SEQ ID NO: 75 - RNA, 19 nt, synthetic construct
  source 1..19: mol_type = other RNA
  modified_base 1..19: mod_base = OTHER; note = phosphorothioate bond
  modified_base 14..17: mod_base = OTHER; note = locked nucleic acid
  modified_base 18..19: mod_base = OTHER; note = 2'-O-methyl
  modified_base 1..13: mod_base = OTHER; note = 2'-O-methyl
atgacaggcattctcatca

SEQ ID NO: 76 - RNA, 19 nt, synthetic construct
  source 1..19: mol_type = other RNA
  modified_base 1..19: mod_base = OTHER; note = phosphorothioate bond
  modified_base 1..2: mod_base = OTHER; note = locked nucleic acid
  modified_base 19: mod_base = OTHER; note = locked nucleic acid
  modified_base 3..18: mod_base = OTHER; note = 2'-O-methyl
atgacaggcattctcatca

SEQ ID NO: 77 - RNA, 19 nt, synthetic construct
  source 1..19: mol_type = other RNA
  modified_base 1..19: mod_base = OTHER; note = phosphorothioate bond
  modified_base 1..2: mod_base = OTHER; note = locked nucleic acid
  modified_base 19: mod_base = OTHER; note = locked nucleic acid
  modified_base 3..18: mod_base = OTHER; note = 2-O-methoxyethyl
atgacaggcattctcatca

SEQ ID NO: 78 - RNA, 19 nt, synthetic construct
  source 1..19: mol_type = other RNA
  modified_base 1..19: mod_base = OTHER; note = phosphorothioate bond
  modified_base 16: mod_base = OTHER; note = locked nucleic acid
  modified_base 1..2: mod_base = OTHER; note = locked nucleic acid
  modified_base 17..19: mod_base = OTHER; note = 2'-O-methyl
  modified_base 3..15: mod_base = OTHER; note = 2'-O-methyl
atgacaggcattctcatca

SEQ ID NO: 79 - RNA, 19 nt, synthetic construct
  source 1..19: mol_type = other RNA
  modified_base 1..19: mod_base = OTHER; note = phosphorothioate bond
  modified_base 16: mod_base = OTHER; note = locked nucleic acid
  modified_base 1..2: mod_base = OTHER; note = locked nucleic acid
  modified_base 17..19: mod_base = OTHER; note = 2-O-methoxyethyl
  modified_base 3..15: mod_base = OTHER; note = 2-O-methoxyethyl
atgacaggcattctcatca

SEQ ID NO: 80 - RNA, 19 nt, synthetic construct
  source 1..19: mol_type = other RNA
  modified_base 1..19: mod_base = OTHER; note = phosphorothioate bond
  modified_base 1..2: mod_base = OTHER; note = locked nucleic acid
  modified_base 17: mod_base = OTHER; note = locked nucleic acid
  modified_base 18..19: mod_base = OTHER; note = 2'-O-methyl
  modified_base 3..16: mod_base = OTHER; note = 2'-O-methyl
atgacaggcattctcatca

SEQ ID NO: 81 - RNA, 19 nt, synthetic construct
  source 1..19: mol_type = other RNA
  modified_base 1..19: mod_base = OTHER; note = phosphorothioate bond
  modified_base 1..2: mod_base = OTHER; note = locked nucleic acid
  modified_base 17: mod_base = OTHER; note = locked nucleic acid
  modified_base 18..19: mod_base = OTHER; note = 2-O-methoxyethyl
  modified_base 3..16: mod_base = OTHER; note = 2-O-methoxyethyl
atgacaggcattctcatca

SEQ ID NO: 82 - RNA, 19 nt, synthetic construct
  source 1..19: mol_type = other RNA
  modified_base 1..19: mod_base = OTHER; note = phosphorothioate bond
  modified_base 1: mod_base = OTHER; note = locked nucleic acid
  modified_base 18..19: mod_base = OTHER; note = locked nucleic acid
  modified_base 2..17: mod_base = OTHER; note = 2'-O-methyl
atgacaggcattctcatca

SEQ ID NO: 83 - RNA, 19 nt, synthetic construct
  source 1..19: mol_type = other RNA
  modified_base 1..19: mod_base = OTHER; note = phosphorothioate bond
  modified_base 1: mod_base = OTHER; note = locked nucleic acid
  modified_base 18..19: mod_base = OTHER; note = locked nucleic acid
  modified_base 2..17: mod_base = OTHER; note = 2-O-methoxyethyl
atgacaggcattctcatca

SEQ ID NO: 84 - RNA, 19 nt, synthetic construct
  source 1..19: mol_type = other RNA
  modified_base 1..19: mod_base = OTHER; note = phosphorothioate bond
  modified_base 1: mod_base = OTHER; note = locked nucleic acid
  modified_base 19: mod_base = OTHER; note = locked nucleic acid
  modified_base 17: mod_base = OTHER; note = locked nucleic acid
  modified_base 2..16: mod_base = OTHER; note = 2'-O-methyl
  modified_base 18: mod_base = OTHER; note = 2'-O-methyl
atgacaggcattctcatca

SEQ ID NO: 85 - RNA, 19 nt, synthetic construct
  source 1..19: mol_type = other RNA
  modified_base 1..19: mod_base = OTHER; note = phosphorothioate bond
  modified_base 1: mod_base = OTHER; note = locked nucleic acid
  modified_base 19: mod_base = OTHER; note = locked nucleic acid
  modified_base 17: mod_base = OTHER; note = locked nucleic acid
  modified_base 2..16: mod_base = OTHER; note = 2-O-methoxyethyl
  modified_base 18: mod_base = OTHER; note = 2-O-methoxyethyl
atgacaggcattctcatca

SEQ ID NO: 86 - RNA, 19 nt, synthetic construct
  source 1..19: mol_type = other RNA
  modified_base 1..19: mod_base = OTHER; note = phosphorothioate bond
  modified_base 2: mod_base = OTHER; note = locked nucleic acid
  modified_base 19: mod_base = OTHER; note = locked nucleic acid
  modified_base 17: mod_base = OTHER; note = locked nucleic acid
  modified_base 1: mod_base = OTHER; note = 2'-O-methyl
  modified_base 3..16: mod_base = OTHER; note = 2'-O-methyl
  modified_base 18: mod_base = OTHER; note = 2'-O-methyl
atgacaggcattctcatca

SEQ ID NO: 87 - RNA, 19 nt, synthetic construct
  source 1..19: mol_type = other RNA
  modified_base 1..19: mod_base = OTHER; note = phosphorothioate bond
  modified_base 2: mod_base = OTHER; note = locked nucleic acid
  modified_base 19: mod_base = OTHER; note = locked nucleic acid
  modified_base 17: mod_base = OTHER; note = locked nucleic acid
  modified_base 1: mod_base = OTHER; note = 2-O-methoxyethyl
  modified_base 3..16: mod_base = OTHER; note = 2-O-methoxyethyl
  modified_base 18: mod_base = OTHER; note = 2-O-methoxyethyl
atgacaggcattctcatca